(19) United States
(12) Patent Application Publication
(10) Pub. No.: US 2026/0241050 A1
(43) Pub. Date: Aug. 20, 2026

(54) REAGENTS AND METHODS FOR IMAGING CANCERS EXPRESSING PDGFRALPHA

(71) Applicants: Telix Pharmaceuticals (Innovations) Pty Ltd., Melbourne (AU); Eli Lilly and Company, Indianapolis, IN (US)

(72) Inventors: Michael Paul Wheatcroft, North Melbourne (AU); Alesia Ivashkevich, North Melbourne (AU); Corinne Letendre, North Melbourne (AU); Rodney John Hicks, North Melbourne (AU); Tracey Jean Brown, North Melbourne (AU); Neel Jagdish Patel, North Melbourne (AU); Angela Man Yin Hong, North Melbourne (AU)

(73) Assignees: Telix Pharmaceuticals (Innovations) Pty Ltd., Melbourne (AU); Eli Lilly and Company, Indianapolis, IN (US)

(21) Appl. No.: 19/472,083

(22) PCT Filed: Apr. 4, 2024
(86) PCT No.: PCT/AU2024/050322

§ 371 Date: Oct. 2, 2025

Related U.S. Application Data
(60) Provisional application No. 63/494,160, filed on Apr. 4, 2023.

(30) Foreign Application Priority Data
Apr. 4, 2023 (WO) PCT/US2023/050263

(51) Int. Cl. A61K 51/10; A61P 35/00; C07K 16/28

(52) U.S. Cl. CPC A61K 51/1096; A61K 51/103; A61P 35/00; C07K 16/2863; A61K 2121/00; C07K 2317/21; C07K 2317/56; C07K 2317/92

(57) ABSTRACT

The invention relates to reagents for in vivo imaging and detection of cancers that express platelet derived growth factor receptor alpha (PDGFRalpha) using an olaratumab antibody, and methods of use thereof including methods for imaging cancers and methods for selecting patients for treatment.

### RELATED APPLICATIONS

This application claims priority from U.S. provisional application No. 63/494,160 filed 4 Apr. 2023, and International application no, PCT/AU2023/050263 filed 4 Apr. 2023, the entire contents of which are hereby incorporated by reference.

### FIELD OF THE INVENTION

The invention relates to reagents for in vivo imaging and detection of cancers that express platelet derived growth factor receptor alpha (PDGFRalpha), and methods of use thereof including methods for imaging cancers and methods for selecting patients for treatment.

### BACKGROUND OF THE INVENTION

Methods of medical imaging are often used to assist in the diagnosis and staging of progression of various cancers. Such methods may be advantageous in removing or reducing the need for invasive techniques (such as obtaining a biopsy sample) for confirming diagnosis, which are not always necessary and can lead to complications.

Not all cancers can be successfully detected using standard medical imaging. Moreover, many imaging techniques enable the detection of masses but cannot successfully distinguish between benign or malignant tissue.

Cancer is a heterogeneous disease with wide variations in tumour morphology and physiology. Although some conventional histologic and clinical features have been correlated to prognosis, the vast heterogeneity across the forms of cancer, spanning from the cellular to the tissue level, impacts response to therapy and subsequent benefit to the patient. Therefore, selectively treating cancer patients who will benefit from a particular treatment continues to pose a challenge.

There is a need for improved methods and compositions for use in the in vivo detection and/or imaging of various cancers. There is a need for improved methods and compositions for determining the likelihood that a patient will benefit from a particular cancer treatment.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present invention relates to agents and methods for detecting, imaging and diagnosing cancers which express platelet derived growth factor receptor alpha (PDGFRalpha); and methods for identifying subjects for treatment with an inhibitor of PDGFRalpha.

In a first aspect, the present invention provides an olaratumab antibody or antigen binding fragment thereof, wherein the antibody or antigen binding fragment thereof is conjugated to a radioisotope, for:
the detection or imaging of PDGFRalpha-expressing tumours in a subject;
for the diagnosis of a PDGFRalpha-expressing cancer in a subject;
for producing an image of a PDGFRalpha-expressing cancer in a subject;
for classifying a cancer as sensitive to treatment with a PDGFRalpha-inhibitor;
for classifying or selecting a patient for eligibility for cancer therapy with an inhibitor of PDGFRalpha; or
for monitoring the response of a PDGFRalpha-expressing cancer or a subject having a PDGFRalpha-expressing cancer, to treatment.

It will be understood that an olaratumab antibody or antigen binding fragment thereof, wherein the antibody or antigen binding fragment thereof is conjugated to a radioisotope, may also be referred to herein as a radiolabelled olaratumab antibody or antigen binding fragment thereof.

In a second aspect, the present invention provides an olaratumab antibody or olaratumab antibody bioconjugate, suitable for radiolabelling with a diagnostic radioisotope.

Preferably the olaratumab bioconjugate comprises an olaratumab antibody conjugated to any chelating or linker group suitable for further conjugation to a radioisotope. Optionally the olaratumab bioconjugate is selected from: olaratumab-TMT (6,6″-bis[N,N″,N′″-tetra(carboxymethyl)aminomethyl)-4′-(3-amino-4-methoxyphenyl)-2,2′:6′,2″-terpyridine), olaratumab-DOTA (1,4,7,10-tetraazacyclododecane-NN′,N″(N′″-tetraacetic acid), olaratumab-TCMC, olaratumab-DO3A, olaratumab-CB-DO2A, olaratumab-NOTA, olaratumab-Diamsar, olaratumab-DTPA, olaratumab-CHX-A″-DTPA, olaratumab-TETE, olaratumab-Te2A, olaratumab-HBED, olaratumab-DFO, olaratumab-DFOsq, olaratumab-DFO-NCS and olaratumab-HOPO or olaratumab chelated to a chelator as disclosed in WO 2022/133537 or to any other chelator as described herein or known to the skilled person.

The olaratumab antibody or antigen binding fragment thereof preferably comprises an antigen binding domain that competitively inhibits the binding of an antibody comprising a VH comprising a sequence as set forth in SEQ ID NO: 4 and a VL comprising a sequence as set forth in SEQ ID NO: 12.

In any aspect or embodiment herein, the olaratumab antibody or antigen binding fragment thereof comprises an HCDR1, a HCDR2 and an HCDR3 of an antigen binding domain having a variable heavy chain as defined in in SEQ ID NO: 4 and a LCDR1, LCDR2 and LCDR3 of an antigen binding domain having a VL as defined in SEQ ID NO: 12.

As used herein, the complementarity determining region sequences (CDRs) of an antigen binding protein of the invention may be defined according to the IMGT, Chothia or Kabat numbering systems, or any other CDR numbering system known to the skilled person.

In any aspect or embodiment of the invention, the olaratumab antibody or antigen binding fragment thereof comprises an antigen binding domain comprising:
(i) a VH comprising a complementarity determining region (CDR) 1 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical to a sequence set forth in SEQ ID NO: 1, a CDR2 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical to a sequence set in SEQ ID NO: 2, and a CDR3 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical to a sequence set forth in SEQ ID NO: 3;
(ii) a VH comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical to a sequence set forth in SEQ ID NO: 4;
(iii) a VL comprising a CDR1 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical to a sequence set forth in SEQ ID NO: 9, a CDR2 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical to a sequence set forth in SEQ ID NO: 10 and a CDR3 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical to a sequence set forth in SEQ ID NO: 11;
(iv) a VL comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical to a sequence set forth in SEQ ID NO: 12;
(v) a VH comprising a CDR1 comprising a sequence set forth in SEQ ID NO: 1, a CDR2 comprising a sequence set forth in SEQ ID NO: 2 and a CDR3 comprising a sequence set forth in SEQ ID NO: 3;
(vi) a VH comprising a sequence set forth in SEQ ID NO: 4;
(vii) a VL comprising a CDR1 comprising a sequence set forth in SEQ ID NO: 9, a CDR2 comprising a sequence set forth in SEQ ID NO: 10 and a CDR3 comprising a sequence set forth in SEQ ID NO: 11;
(viii) a VL comprising a sequence set forth in SEQ ID NO: 12;
(ix) a VH comprising a CDR1 comprising a sequence set forth in SEQ ID NO: 1, a CDR2 comprising a sequence set forth between in SEQ ID NO: 2, and a CDR3 comprising a sequence set forth in SEQ ID NO: 3; and a VL comprising a CDR1 comprising a sequence set SEQ ID NO: 9, a CDR2 comprising a sequence set forth in SEQ ID NO: 10, and a CDR3 comprising a sequence set forth in SEQ ID NO: 11; or
(x) a VH comprising a sequence set forth in SEQ ID NO: 4, and a VL comprising a sequence set forth in SEQ ID NO: 12.

The olaratumab antibody or antigen binding fragment thereof may further comprise:
(i) a VH comprising a framework region (FR) 1 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in SEQ ID NO: 5; a FR2 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in SEQ ID NO: 6; a FR3 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in SEQ ID NO: 7; a FR4 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in SEQ ID NO: 8; and
(ii) a VL comprising a framework region (FR) 1 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in SEQ ID NO: 13; a FR2 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in SEQ ID NO: 14; a FR3 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in SEQ ID NO: 15; a FR4 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in SEQ ID NO: 16.

In a further embodiment, the olaratumab antibody or antigen binding fragment thereof comprises:
(i) a VH comprising a framework region (FR) 1 comprising or consisting of a sequence as set forth in SEQ ID NO: 5; a FR2 comprising or consisting of a sequence as set forth in SEQ ID NO: 6; a FR3 comprising or consisting of a sequence as set forth in SEQ ID NO: 7; a FR4 comprising or consisting of a sequence as set forth in SEQ ID NO: 8, and
(ii) a VL comprising a framework region (FR) 1 comprising or consisting of a sequence as set forth in SEQ ID NO: 13; a FR2 comprising or consisting of a sequence as set forth in SEQ ID NO: 14; a FR3 comprising or consisting of a sequence as set forth in SEQ ID NO: 15; a FR4 comprising or consisting of a sequence as set forth in SEQ ID NO: 16.

In any embodiment, the olaratumab antibody or antigen binding fragment thereof comprises a VH comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical to a sequence set forth in SEQ ID NO: 4; and/or a VL comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical to a sequence set forth in SEQ ID NO: 12; wherein the sequence variation between the VH and VL and the sequence of SEQ ID NO: 4 and 12 is not in the CDRs and wherein the antigen binding protein retains the ability to bind to PDGFRalpha.

In any embodiment, the olaratumab antibody or antigen binding fragment thereof comprises a VH and/or a VL comprising no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19 or no more than 20 amino acid residue substitutions, deletions or additions, compared to the amino acid sequences as set forth in SEQ ID NO: 4 or 12, respectively; wherein the amino acid substitutions, deletions or additions are not in the CDRs and the antigen binding protein retains the ability to bind to PDGFRalpha.

As used herein, the terms HCDR1, HCDR2 and HCDR3 will be understood to refer to the CDRs of the variable heavy chain; the terms LCDR1, LCDR2, and LCDR3, will be understood to refer to the CDRs of the variable light chain; and the terms HFR1, HFR2, HFR3, HFR4, LFR1, LFR2, LFR3 and LFR4 will be understood to refer to the framework regions of the heavy, and light chains respectively.

As described herein, the olaratumab antibody or antigen binding fragment thereof may be in the form of:
(i) a single domain antibody (sdAb);
(ii) a single chain Fv fragment (scFv);
(iii) a dimeric scFv (di-scFv); or
(iv) one of (ii) or (iii) linked to a constant region of an antibody, Fc or a heavy chain constant domain (CH) 2 and/or CH3.

Further, as described herein, the olaratumab or antigen binding fragment thereof may be in the form of:
(i) a diabody;
(ii) a triabody;
(iii) a tetrabody;
(iv) a Fab;
(v) a F(ab′)2;
(vi) a Fv;
(vii) a bispecific antibody or other form of multispecific antibody; or
(viii) one of (i) to (vii) linked to a constant region of an antibody, Fc or a heavy chain constant domain (CH) 2 and/or CH3.

Optionally, the variable heavy and variable light regions of the antigen binding domain are joined via a linker.

In any embodiment, the antigen binding domain may comprise:

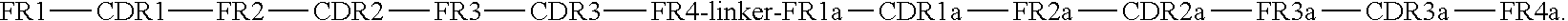

As defined herein, the linker may be a chemical, one or more amino acids, or a disulphide bond formed between two cysteine residues.

In any aspect or embodiment of the present invention, the radiolabelled-olaratumab or antigen binding fragment thereof may comprise a human constant region, e.g., an IgG constant region, such as an IgG₁, IgG₂, IgG₃ or IgG₄ constant region or mixtures thereof. In the case of an antibody or protein comprising a VH and a VL, the VH can be linked to a heavy chain constant region and the VL can be linked to a light chain constant region.

In one example, the olaratumab or antigen binding fragment thereof comprises a constant region of an IgG₄ antibody or a stabilised constant region of an IgG₄ antibody. In one example, the olaratumab or antigen binding fragment thereof comprises an IgG₄ constant region with a proline at position 241 (according to the numbering system of Kabat (Kabat et al., Sequences of Proteins of Immunological Interest Washington DC United States Department of Health and Human Services, 1987 and/or 1991)).

In one example, the olaratumab or antigen binding fragment thereof comprises a heavy chain constant region, comprising a stabilised heavy chain constant region, comprising a mixture of sequences fully or partially with or without the C-terminal lysine residue.

In further embodiments of any aspect herein, the olaratumab or antigen binding fragment thereof comprises an Fc region wherein the Fc region is engineered to have enhanced capacity to induce antibody-dependent cell-mediated cytotoxicity (ADCC). Preferably, the enhanced capacity to induce ADCC is conferred by mutation, deletion or modification of amino acids in the Fc region which interact with an Fc receptor

In another embodiment, the olaratumab or antigen binding fragment thereof comprises an Fc region that is engineered to:
have increased in vitro or in vivo half-life;
have an increased capacity to induce antibody-dependent cell mediated cytotoxicity (ADCC), antibody-dependent cellular phagocytosis (ADCP) or complement-dependent cytotoxicity; and/or
have reduced effector function.

Preferably, the olaratumab antibody is in the form of an antibody (i.e. comprising a variable light and variable heavy chain, linked to a constant region of an antibody including a heavy chain CH2 and/or CH3).

In further embodiments, the olaratumab may comprise a heavy chain constant region as defined in SEQ ID NO: 17 and/or a light chain constant region as defined in SEQ ID NO: 18.

In particularly preferred embodiments, the olaratumab may comprise a heavy chain as set forth in SEQ ID NO: 19 and/or a light chain as set forth in SEQ ID NO: 20.

In still further embodiments, the olaratumab is in the form of an antibody and may comprise one or more amino acid substitutions in the constant regions, so as to reduce the in vivo half-life of the antibody. The present invention therefore provides for an olaratumab antibody having substitutions in the CH2 and/or CH3 domains of the constant region and comprising substitutions at one or more of residues His310, His435, Tyr436 and Ile253 (Kabat EU index numbering), thereby altering FcRn binding affinity and/or serum half-life of said antibody (e.g. relative to an olaratumab that comprises a native CH2 and CH3 domain).

In some examples, the amino acid at position 310 and/or 435 of the antibody may be alanine, glutamic acid, aspartic acid, leucine, isoleucine, arginine, proline, glutamine, methionine, serine, threonine, lysine, asparagine, phenylalanine, tyrosine, tryptophan, cysteine, valine or glycine.

Preferably, the residue at position 310 is selected from alanine, or glutamic acid or glutamine; or amino acid residue 435 from the heavy chain constant region is selected from arginine, glutamine or alanine. In other preferred embodiments, the antibody has an alanine residue at position 310 and glutamine residue at position 435.

In a further embodiment, the antibody also comprises an amino acid substitution at residue Lys322. Preferably the substitution is K322A.

In particularly preferred embodiments, the olaratumab antibody comprises substitutions K322A, H31 OA and H435Q.

In another preferred embodiment, the olaratumab antibody comprises amino acid substitutions at residues His310, His435 and Ile253, wherein preferably the substitutions are H310A, H435Q and 1253E.

In a preferred embodiment, the binding affinity for FcRn and/or the serum half-life of the modified antibody is decreased by at least about 2-fold, 3-fold, 4-fold, 5-fold, 10-fold, 15-fold, 20-fold, 25-fold, 30-fold, 40-fold, 50-fold, 60-fold, 70-fold, 80-fold, 90-fold, or 100-fold. In a preferred embodiment of the present invention, the binding affinity for FcRn and/or the serum half-life of said modified antibody is reduced by at least about 20%, 30%, 40%, 50%, 60%, 70%, 80%, 85%, 90%, 95%, 97%, 98%, or 99%.

In any aspect or embodiment, the antibody may also comprise amino acid substitutions at residues equivalent to Ser228 and/or Leu235 of the constant heavy chain region, such as Ser228Pro and/or Leu235Glu.

In any aspect or embodiment, the radiolabelled olaratumab may comprise any radioisotope suitable for in vivo imaging methods for detection of tumour or cancer masses. Examples of suitable radioisotopes include: fluorine-18 (¹⁸F), gallium-67 and gallium-68 (⁶⁷Ga and ⁶⁸Ga), indium-111 (¹¹¹In), iodine-123 and iodine-124, technetium-99 (^{99m}Tc) terbium-155 and terbium-159 (¹⁵⁵Tb and ¹⁵⁹Tb) and zirconium-89 (⁸⁹Zr).

In any aspect or embodiment, the radioisotope may be conjugated to the olaratumab directly e.g. by halogenation of amino acid residues. Preferably, the radioisotope of the radiolabelled olaratumab is linked indirectly to the olaratumab or antigen binding fragment thereof, for example via a chelator or other linking moiety. In one example, the olaratumab is conjugated to a chelating moiety, selected from the group consisting of: TMT (6,6″-bis[N,N″,N″-tetra(carboxymethyl)aminomethyl)-4′-(3-amino-4-methoxyphenyl)-2,2′:6′,2″-terpyridine), DOTA (1, 4,7,10-tetraazacyclododecane-NN′,N″(N′″-tetraacetic acid), TCMC, DO3A, CB-DO2A, NOTA, Diamsar, DTPA, CHX-A″-DTPA, TETE, Te2A, HBED, DFO, DFOsq, DFO-NCS and HOPO, a chelating agent as described in WO 2022/133537 (incorporated herein by reference),or other chelating agent as described herein or known to the skilled person.

In another example, the radiolabelled olaratumab or antigen binding fragment thereof is conjugated to a bifunctional linker, for example, bromoacetyl, thiols, succinimide ester, TFP ester, a maleimide, or using any amine or thiol-modifying chemistry known in the art.

Preferably the radiolabelled olaratumab antibody or antigen binding fragment thereof is ⁸⁹Zr-olaratumab or a ⁸⁹Zr labelled olaratumab, preferably wherein the ⁸⁹Zr is conjugated to the olaratumab via a linker, such as ⁸⁹Zr-DFO-olaratumab, ⁸⁹Zr-DFO-NCS-olaratumab, or ⁸⁹Zr-DFO-Sq-olaratumab.

In a further aspect there is provided a method for obtaining a radiolabelled olaratumab of the first aspect of the invention, wherein the method comprises radiolabelling an olaratumab bioconjugate or antibody of the second aspect.

In a second aspect, the present invention provides a method for in vivo imaging or detection of a PDGFRalpha-expressing cancer in a subject, wherein the method comprises:
administering to a subject in need thereof, a radiolabelled-olaratumab antibody or antigen binding fragment thereof, preferably wherein the radiolabelled-olaratumab is as described herein,
detecting the antibody or antigen binding fragment thereof in the subject.

In a third aspect, the present invention provides a method for the diagnosis of a PDGFRalpha-expressing cancer in a subject, where the method comprises:
administering to a subject in need thereof, a radiolabelled-olaratumab antibody or antigen binding fragment thereof, preferably wherein the radiolabelled-olaratumab is as described herein,
determining the presence or absence of the antibody or antigen binding fragment thereof in the subject.

In accordance with the second or third aspects of the invention, the detection of the antibody or fragment, or determining the presence or absence of the antibody or fragment, may be compared to a background or standard level to determine the presence of the cancer, thereby imaging or detecting the cancer in the subject.

Alternatively, or in addition, the detection or the presence or absence is compared to a model of disease classification or disease progression that is derived from data from one or more individuals, thereby imaging or detecting or diagnosing the cancer in the subject. The model may be derived from individuals who are known to have the cancer or from individuals who are known not to have the cancer (or combinations thereof).

In certain embodiments, the detecting or determining may result in data which is used to compile training data for development of a deep learning algorithm for enabling artificial-intelligence based self-learning for diagnosis and/or staging of the cancer. Such methods are generally known in the art and are described for example in WO2020144134 and U.S. Pat. No. 11,443,201, incorporated herein by reference.

The present invention also provides a method for producing an image of a PDGFRalpha-expressing cancer in a subject, the method comprising:
administering to a subject suspected of having the cancer, a radiolabelled-olaratumab antibody or antigen binding fragment thereof, preferably wherein the radiolabelled-olaratumab is as described herein,
detecting the antibody or antigen binding fragment thereof in the subject, thereby producing an image of the cancer.

The present invention provides a method for producing an image of a PDGFRalpha-expressing cancer, the method comprising:
infusing an effective amount of a radiolabelled-olaratumab antibody or antigen binding fragment thereof, preferably wherein the radiolabelled-olaratumab is as described herein,
detecting the antibody or antigen binding fragment thereof, thereby producing an image of the cancer.

Optionally the image of the cancer produced is used to generate training data for machine learning algorithm development.

In a further aspect, the present invention provides a method for classifying a cancer as sensitive to treatment with an inhibitor of PDGFRalpha, the method comprises:
administering to a subject in need thereof, a radiolabelled-olaratumab antibody or antigen binding fragment thereof, preferably wherein the radiolabelled-olaratumab is as described herein,
detecting the antibody or antigen binding fragment thereof in the subject,
   whereby the cancer is classified as sensitive to treatment with an inhibitor of PDGFRalpha when detection of the antibody or fragment thereof is below or the same as a background or threshold level; or
   whereby the cancer is classified as not being sensitive to treatment with an inhibitor of PDGFRalpha when detection of the antibody or fragment thereof is above a background or threshold level.

In a further aspect, the present invention provides a method for classifying a cancer as sensitive to treatment with an inhibitor of PDGFRalpha, the method comprises:
administering to a subject in need thereof, a radiolabelled-olaratumab antibody or antigen binding fragment thereof, preferably wherein the radiolabelled-olaratumab is as described herein,
detecting the antibody or antigen binding fragment thereof in the subject,
   whereby the cancer is classified as sensitive to treatment with an inhibitor of PDGFRalpha based on the output of a deep learning algorithm, indicating the presence of PDGFRalpha expressing cancer cells in the subject,
   whereby the cancer is not classified as sensitive to treatment with an inhibitor of PDGFRalpha based on the output of a deep learning algorithm, indicating the absence of PDGFRalpha expressing cancer cells in the subject,

In a further aspect, the present invention provides a method for classifying or selecting a patient for eligibility for cancer therapy with an inhibitor of PDGFRalpha, the method comprising:
administering to a subject in need thereof, a radiolabelled-olaratumab antibody or antigen binding fragment thereof, preferably wherein the radiolabelled-olaratumab is as described herein,
detecting the antibody or antigen binding fragment thereof in the subject,
   whereby the patient is classified/selected for cancer therapy with an inhibitor of PDGFRalpha when detection of the antibody or fragment thereof is above a background or standard level;
   whereby the patient is not classified/selected for cancer therapy with an inhibitor of PDGFRalpha when detection of the antibody or fragment thereof is the same or below a background or standard level.

In a further aspect, the present invention provides a method for classifying or selecting a patient for eligibility for cancer therapy with an inhibitor of PDGFRalpha, the method comprising:
administering to a subject in need thereof, a radiolabelled-olaratumab antibody or antigen binding fragment thereof, preferably wherein the radiolabelled-olaratumab is as described herein,
detecting the antibody or antigen binding fragment thereof in the subject,
   whereby the patient is classified/selected for cancer therapy with an inhibitor of PDGFRalpha based on the output of a deep learning algorithm, indicating the presence of PDGFRalpha expressing cancer cells in the subject,
   whereby the patient is not classified/selected for cancer therapy with an inhibitor of PDGFRalpha based on the output of a deep learning algorithm, indicating the absence of PDGFRalpha expressing cancer cells in the subject.

In a further aspect, the present invention relates to methods for monitoring the response to a treatment of a PDGFRalpha-expressing cancer. As such, the invention provides a method for determining the response to treatment of a PDGFRalpha-expressing cancer or a subject having a PDGFRalpha-expressing cancer, the method comprising:
administering to a subject who has received or is receiving a treatment for a PDGFRalpha-expressing cancer, a radiolabelled-olaratumab antibody or antigen binding fragment thereof, preferably wherein the radiolabelled-olaratumab is as described herein,
detecting the antibody or antigen binding fragment thereof in the subject,
comparing the amount of antibody or antigen binding fragment thereof detected in the subject compared to a reference amount of the antibody or antigen binding fragment thereof detected in the subject prior to the treatment,
determining that the cancer has responded to the treatment when the amount of the antibody or antigen binding fragment thereof detected is lower than the reference amount,
determining that the cancer has not responded to the treatment when the amount of the antibody or antigen binding fragment thereof is the same or higher than the reference amount,
   thereby determining the response to treatment of a PDGFRalpha-expressing cancer or a subject having a PDGFRalpha-expressing cancer.

The treatment received or being received by the subject may be any treatment for the PDGFRalpha-expressing cancer, including but not limited to, surgery, chemotherapy, immunotherapy (such as CAR-T therapy), radiotherapy (including external beam radiation or immuno-radiotherapy), an inhibitor of PDGFRalpha and/or combinations thereof.

Further still the present invention provides methods for treating a cancer patient, the method comprising:
administering to a cancer patient suspected of having or considered at risk of having a cancer expressing PDGFRalpha, a radiolabelled-olaratumab antibody or antigen binding fragment thereof, preferably wherein the radiolabelled-olaratumab is as described herein,
detecting the antibody or antigen binding fragment thereof in the patient,
classifying the patient as eligible to receive cancer therapy with an inhibitor of PDGFRalpha, when detection of the antibody or fragment thereof is above a background or standard level;
classifying the patient as ineligible to receive cancer therapy with an inhibitor of PDGFRalpha, when detection of the antibody or fragment thereof is the same or below a background or standard level;
treating the patient with an inhibitor of PDGFRalpha when the patient is classified as eligible to receive cancer therapy with an inhibitor of PDGFRalpha.
determining not to treat the patient with an inhibitor of PDGFRalpha when the patients are classified as ineligible to receive cancer therapy with an inhibitor of PDGFRalpha.

Further still the present invention provides methods for treating a cancer patient, the method comprising:
administering to a cancer patient suspected of having or considered at risk of having a cancer expressing PDGFRalpha, a radiolabelled-olaratumab antibody or antigen binding fragment thereof, preferably wherein the radiolabelled-olaratumab is as described herein,
detecting the antibody or antigen binding fragment thereof in the patient,
classifying the patient as eligible to receive cancer therapy with an inhibitor of PDGFRalpha, based on the output of a deep learning algorithm derived from data compiled from subjects who responded to treatment with an inhibitor of PDGFRalpha;
classifying the patient as ineligible to receive cancer therapy with an inhibitor of PDGFRalpha, based on the output of a deep learning algorithm derived from data compiled from subjects who did not respond to treatment with an inhibitor of PDGFRalpha;
treating the patient with an inhibitor of PDGFRalpha when the patient is classified as eligible to receive cancer therapy with an inhibitor of PDGFRalpha.
determining not to treat the patient with an inhibitor of PDGFRalpha when the patients are classified as ineligible to receive cancer therapy with an inhibitor of PDGFRalpha.

The present invention also provides methods for treating a cohort of cancer patients, the method comprising:
administering to the cohort, a radiolabelled-olaratumab antibody or antigen binding fragment thereof, preferably wherein the radiolabelled-olaratumab is as described herein,
detecting the antibody or antigen binding fragment thereof in the cohort,
classifying patients as eligible to receive cancer therapy with an inhibitor of PDGFRalpha, when detection of the antibody or fragment thereof is above a background or standard level;
classifying patients as ineligible to receive cancer therapy with an inhibitor of PDGFRalpha, when detection of the antibody or fragment thereof is the same or below a background or standard level;
treating patients in the cohort with an inhibitor of PDGFRalpha when the patients are classified as eligible to receive cancer therapy with an inhibitor of PDGFRalpha.
determining not to treat patients in the cohort with an inhibitor of PDGFRalpha when the patients are classified as ineligible to receive cancer therapy with an inhibitor of PDGFRalpha.

The present invention also provides methods for treating a cohort of cancer patients, the method comprising:
administering to the cohort, a radiolabelled-olaratumab antibody or antigen binding fragment thereof, preferably wherein the radiolabelled-olaratumab is as described herein,
detecting the antibody or antigen binding fragment thereof in the cohort,
classifying patients as eligible to receive cancer therapy with an inhibitor of PDGFRalpha, based on the output of a deep learning algorithm derived from data compiled from subjects who responded to treatment with an inhibitor of PDGFRalpha;
classifying patients as ineligible to receive cancer therapy with an inhibitor of PDGFRalpha, based on the output of a deep learning algorithm derived from data compiled from subjects who did not respond to treatment with an inhibitor of PDGFRalpha;
treating patients in the cohort with an inhibitor of PDGFRalpha when the patients are classified as eligible to receive cancer therapy with an inhibitor of PDGFRalpha.
determining not to treat patients in the cohort with an inhibitor of PDGFRalpha when the patients are classified as ineligible to receive cancer therapy with an inhibitor of PDGFRalpha.

In any of the aforementioned aspects or embodiments of the invention, the inhibitor of PDGFRalpha may be an olaratumab antibody, or functional antigen binding fragment thereof. As such, and in accordance with the aforementioned aspects and embodiments, the present invention therefore provides methods for:
classifying a cancer as sensitive to treatment with an olaratumab antibody, or functional antigen binding fragment thereof;
classifying a patient for eligibility for cancer therapy with an olaratumab antibody, or functional antigen binding fragment thereof;
selecting a patient for treatment with an olaratumab antibody, or functional antigen binding fragment thereof; or
treating a patient or cohort of cancer patients with an olaratumab antibody, or functional antigen binding fragment thereof,
   wherein preferably the olaratumab antibody for use in a method described comprises an amino acid sequence as defined herein in Table 1. Most preferably, the olaratumab antibody comprises a heavy variable chain as defined in SEQ ID NO: 4 and a light variable chain as defined in SEQ ID NO: 12.

The invention also provides for a radiolabelled-olaratumab antibody or antigen binding fragment thereof, or a composition comprising the same, as described herein, for use in a method of:
in vivo imaging or detection of a PDGFRalpha-expressing cancer;
diagnosis of a PDGFRalpha-expressing cancer;
producing an image of a PDGFRalpha-expressing cancer;
classifying a cancer as sensitive to treatment with an inhibitor of PDGFRalpha;
classifying or selecting a patient for eligibility for cancer therapy with an inhibitor of PDGFRalpha;
treating a cancer patient or cohort of cancer patients with an inhibitor of PDGFRalpha;
   wherein the methods are as described herein and preferably wherein the radiolabelled-olaratumab is as described herein.

In another aspect, the invention provides a use of a radiolabelled-olaratumab antibody or antigen binding fragment thereof, in the manufacture of a composition for:
in vivo imaging or detection of a PDGFRalpha-expressing cancer;
diagnosis of a PDGFRalpha-expressing cancer;
producing an image of a PDGFRalpha-expressing cancer;
classifying a cancer as sensitive to treatment with an inhibitor of PDGFRalpha;
classifying or selecting a patient for eligibility for cancer therapy with an inhibitor of PDGFRalpha;
treating a cancer patient or cohort of cancer patients with an inhibitor of PDGFRalpha;
   wherein the methods are as described herein and preferably wherein the radiolabelled-olaratumab is as described herein.

Further still, the invention provides for a radiolabelled-olaratumab antibody or antigen binding fragment thereof, or a composition comprising the same, when used in a method of:
in vivo imaging or detection of a PDGFRalpha-expressing cancer;
diagnosis of a PDGFRalpha-expressing cancer;
producing an image of a PDGFRalpha-expressing cancer;
classifying a cancer as sensitive to treatment with an inhibitor of PDGFRalpha;
classifying or selecting a patient for eligibility for cancer therapy with an inhibitor of PDGFRalpha;
treating a cancer patient or cohort of cancer patients with an inhibitor of PDGFRalpha;
   wherein the methods are as described herein, and preferably wherein the radiolabelled-olaratumab is as described herein.

Further still, the invention provides a kit for use in accordance with any method described herein, wherein the kit comprises: and a radiolabelled-olaratumab antibody or antigen binding fragment thereof as described herein, and optionally, instructions for the use thereof for
in vivo imaging or detection of a PDGFRalpha-expressing cancer;
diagnosis of a PDGFRalpha-expressing cancer;
producing an image of a PDGFRalpha-expressing cancer;
classifying a cancer as sensitive to treatment with an inhibitor of PDGFRalpha;
classifying or selecting a patient for eligibility for cancer therapy with an inhibitor of PDGFRalpha;
treating a cancer patient or cohort of cancer patients with an inhibitor of PDGFRalpha.

In a further aspect, there is provided a pharmaceutical composition comprising an olaratumab antibody, or antigen binding fragment thereof, an olaratumab bioconjugate or a radiolabelled olaratumab as herein described, optionally in combination with a pharmaceutically acceptable excipient.

Further still there is provided a nucleic acid or nucleic acid construct encoding an olaratumab antibody, or antigen binding fragment thereof, or an olaratumab bioconjugate as described herein.

There is also provided a host or host cell comprising a nucleic acid or nucleic acid construct of the invention.

In any aspect or embodiment above, the methods of the invention further may comprise allowing the radiolabelled-olaratumab antibody or antigen binding fragment thereof to concentrate at sites and/or tissues in said subject where the PDGFRalpha antigen is found in the subject, prior to the step of detecting.

It will be understood that detection of the radiolabelled-olaratumab antibody or antigen binding fragment thereof preferably comprises determination or detecting the presence or absence of radiation emitted by the radioisotope conjugated to the antibody. In any aspect or embodiment, determination or detection of the presence of absence of radiation comprises Positron Emission Tomography (PET), SPECT imaging or combinations thereof. The methods may be further supplemented with use of CT, MRI or other imaging techniques.

In any aspect or embodiment, a subject in need thereof may be any subject that is suspected of having or is considered at risk of having a PDGFRalpha-expressing cancer. Suspicion of having the cancer, or consideration of risk may be based on any sign or symptom associated with the cancer, a family history of PDGFRalpha-expressing cancers or a genotype associated with PDGFRalpha-expressing cancer or a combination thereof.

In any aspect or embodiment of the invention, the cancer that is detected, imaged, diagnosed, classified or selected may be any cancer expressing PDGFRalpha. Preferably, the cancer is selected from the group consisting of: soft tissue sarcoma (STS), chondrosarcoma, leiomyosarcoma, liposarcoma, osteosarcoma, and rhabdomyosarcoma. Alternatively, the cancer is selected from the group consisting of: gastrointestinal stromal tumour (GIST), non-small cell lung cancer (NSCLC), pancreatic cancer, hepatocellular carcinoma, breast cancer, neuroendocrine tumours, ovarian cancer, cervical cancer, uterine cancer (such as adenosquamous carcinoma of the uterine cervix), colorectal cancer, gastric cancer, salivary gland cancer, biliary cancer, glioma, prostate cancer and renal cell carcinoma.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1: Schematic representation of DFOsq- and DOTA-bioconjugated Olaratumab.

FIG. 2: Set-up of Biacore surface plasmon resonance assay.

FIG. 3: Total and surface PDGFRA expression in KRIB, HuO9 and A-204 sarcoma cell lines. A) Total PDGFRA expression was established with western blot; B) Surface expression of PDGFRα on unfixed cells was determined by FACS.

FIG. 4: Western blot showing olaratumab-mediated inhibition of PDGFRA activation pathway.

FIG. 5: A) Binding of ⁸⁹Zr-DFOSq-Olaratumab to A204 and HCC827 cells at 1 hr; B) Binding of ⁸⁹Zr-DFOSq-Olaratumab and ¹⁷⁷Lu-DOTA-Olaratumab to A204 cells at 1 and 4 hrs.

FIG. 6: Example of the PET images across at 24, 48 and 120 hrs. SUVmax, tumour:background ratio, tumour:liver ratio and tumour:bone ratio was measured for each image.

FIG. 7: Biodistribution of ⁸⁹Zr-DFOsq-Olaratumab at 24, 48 and 120 hrs as measured by radioactivity counts in blood and dissected lungs, heart, liver, kidneys, muscle, spleen, bone and tumour of A204-bearing nude mice.

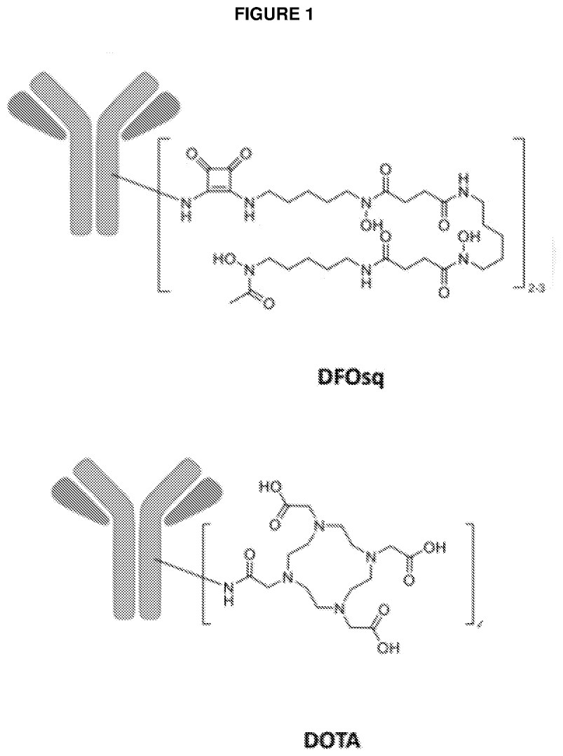

### SEQUENCE INFORMATION

TABLE 1
Summary of amino acid sequences of PDGFRalpha-binding antibodies
(olaratumab)

| Region | SEQ ID NO: | Amino acid or nucleotide sequence |
|---|---|---|
| HCDR1 (protein) | 1 | SSSYY |
| (Kabat) | | |
| HCDR2 (protein) | 2 | SFFYTGSTYYNPSLRS |
| (Kabat) | | |
| HCDR3 (protein) | 3 | QSTYYYGSGNYYGWFDR |
| (Kabat) | | |
| VH (protein) | 4 | QLQLQESGPGLVKPSETLSLTCTVSGGSINSSSYYWGWLRQSPG |
| | | KGLEWIGSFFYTGSTYYNPSLRSRLTISVDTSKNQFSLMLSSVTAA |
| | | DTAVYYCARQSTYYYGSGNYYGWFDRWDQGTLVTVSS |
| HFR1 (protein) | 5 | QLQLQESGPGLVKPSETLSLTCTVSGGSIN |
| (Kabat) | | |
| HFR2 (protein) | 6 | WGWLRQSPGKGLEWIG |
| (Kabat) | | |
| HFR3 (protein) | 7 | RLTISVDTSKNQFSLMLSSVTAADTAVYYCAR |
| (Kabat) | | |
| HFR4 (protein) | 8 | WDQGTLVTVSS |
| (Kabat) | | |
| LCDR1 (protein) | 9 | RASQSVSSYLA |
| (Kabat) | | |
| LCDR2 (protein) | 10 | DASNRAT |
| (Kabat) | | |
| LCDR3 (protein) | 11 | QQRSNWPPA |
| (Kabat) | | |
| VL (protein) | 12 | EIVLTQSPATLSLSPGERATLSCRASQSVSSYLAWYQQKPGQAPR |
| | | LLIYDASNRATGIPARFSGSGSGTDFTLTISSLEPEDFAVYYCQQR |
| | | SNWPPAFGQGTKVEIK |
| LFR1 (protein) | 13 | EIVLTQSPATLSLSPGERATLSC |
| (Kabat) | | |
| LFR2 (protein) | 14 | WYQQKPGQAPRLLIY |
| (Kabat) | | |
| LFR3 (protein) | 15 | GIPARFSGSGSGTDFTLTISSLEPEDFAVYYC |
| (Kabat) | | |
| LFR1 (protein) | 16 | FGQGTKVEIK |
| (Kabat) | | |
| Heavy chain | 17 | ASTKGPSVFPLAPSSKSTSGGTAALGCLVKDYFPEPVTVSWNSG |
| constant region | | ALTSGVHTFPAVLQSSGLYSLSSVVTVPSSSLGTQTYICNVNHKPS |
| | | NTKVDKRVEPKSCDKTHTCPPCPAPELLGGPSVFLFPPKPKDTLM |
| | | ISRTPEVTCVVVDVSHEDPEVKFNWYVDGVEVHNAKTKPREEQY |
| | | NSTYRVVSVLTVLHQDWLNGKEYKCKVSNKALPAPIEKTISKAKG |
| | | QPREPQVYTLPPSREEMTKNQVSLTCLVKGFYPSDIAVEWESNG |
| | | QPENNYKTTPPVLDSDGSFFLYSKLTVDKSRWQQGNVFSCSVMH |
| | | EALHNHYTQKSLSLSPGK |
| Light chain | 18 | RTVAAPSVFIFPPSDEQLKSGTASVVCLLNNFYPREAKVQWKVDN |
| constant region | | ALQSGNSQESVTEQDSKDSTYSLSSTLTLSKADYEKHKVYACEVT |
| | | HQGLSSPVTKSFNRGEC |
| Olaratumab | 19 | QLQLQESGPGLVKPSETLSLTCTVSGGSINSSSYYWGWLRQSPG |
| heavy chain | | KGLEWIGSFFYTGSTYYNPSLRSRLTISVDTSKNQFSLMLSSVTAA |
| | | DTAVYYCARQSTYYYGSGNYYGWFDRWDQGTLVTVSSASTKGP |
| | | SVFPLAPSSKSTSGGTAALGCLVKDYFPEPVTVSWNSGALTSGVH |
| | | TFPAVLQSSGLYSLSSVVTVPSSSLGTQTYICNVNHKPSNTKVDK |
| | | RVEPKSCDKTHTCPPCPAPELLGGPSVFLFPPKPKDTLMISRTPE |
| | | VTCVVVDVSHEDPEVKFNWYVDGVEVHNAKTKPREEQYNSTYRV |
| | | VSVLTVLHQDWLNGKEYKCKVSNKALPAPIEKTISKAKGQPREPQ |
| | | VYTLPPSREEMTKNQVSLTCLVKGFYPSDIAVEWESNGQPENNY |
| | | KTTPPVLDSDGSFFLYSKLTVDKSRWQQGNVFSCSVMHEALHNH |
| | | YTQKSLSLSPGK |
| Olaratumab light | 20 | EIVLTQSPATLSLSPGERATLSCRASQSVSSYLAWYQQKPGQAPR |
| chain | | LLIYDASNRATGIPARFSGSGSGTDFTLTISSLEPEDFAVYYCQQR |
| | | SNWPPAFGQGTKVEIKRTVAAPSVFIFPPSDEQLKSGTASVVCLL |
| | | NNFYPREAKVQWKVDNALQSGNSQESVTEQDSKDSTYSLSSTLT |
| | | LSKADYEKHKVYACEVTHQGLSSPVTKSFNRGEC |

### DETAILED DESCRIPTION OF THE EMBODIMENTS

### Platelet Derived Growth Factor Receptor Alpha (PDGFRalpha)

Platelet derived growth factor receptor alpha (PDGFRα or PDGFRalpha) is a type III receptor tyrosine kinase. PDGFRα is critical for development and fulfills important functions into adulthood. For example, mice homozygous for a null mutation die during embryogenesis. At later stages of development, PDGFRα is expressed in many mesenchymal structures, whereas adjacent epithelial cells produce platelet derived growth factors (PDGFs).

The platelet-derived growth factor family of growth factors consists of five different disulphide-linked dimers, PDGF-AA, -BB, -AB, -CC, and -DD, that act via PDGFRα and PDGFRβ. These growth factors are dimeric molecules composed of disulfide-linked polypeptide chains that bind to two receptor proteins simultaneously and induce receptor dimerization, autophosphorylation, and intracellular signaling. PDGFRα can form heterodimers with PDGFRβ as well as homodimers. Because PDGFRβ does not bind the PDGF-A chain with high affinity, PDGF-AA activates only aa receptor dimers, whereas PDGF-AB and PDGF-CC activates αα and αβ receptor heterodimers.

PDGFR-alpha has also been detected on some tumour and stromal cells, including sarcomas, where signalling can contribute to cancer cell proliferation, metastasis, and maintenance of the tumour microenvironment.

Olaratumab (Lartruvo™) is a fully human IgG₁ monoclonal antibody which selectively binds human Platelet-derived growth factor (PDGF) receptor a (PDGFRα). The interaction between olaratumab and PDGFR-α prevents binding of the receptor by the PDGF-AA and -BB ligands as well as PDGF-AA, -BB, and -CC-induced receptor activation and downstream PDGFR-α pathway signalling. Olaratumab exhibits in vitro and in vivo anti-tumor activity against selected sarcoma cell lines and disrupted the PDGFR-α signaling pathway in in vivo tumor implant models.

In 2019, Lartruvo™ failed to meet the primary overall survival endpoint in the confirmatory phase III trial in soft tissue sarcoma patients (ANNOUNCE trial; NCT02451943). Consequently there is a need to develop new methods for identifying patients who are sensitive to treatment with olaratumab.

The present invention is based on the finding by the inventors that radiolabelled-olaratumab, or antigen binding fragments thereof, can be used to recognise PDGFRα expressed on the surface of soft tissue sarcoma (STS) cells and thereby enable detection and imaging of such cancer cells, as well as identifying patients who are most likely to benefit from therapeutic treatment with PDGFRα inhibitors, such as olaratumab.

### General Definitions

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects, and vice versa, unless the context clearly dictates otherwise. For example, reference to “a” includes a single as well as two or more; reference to “an” includes a single as well as two or more; reference to “the” includes a single as well as two or more and so forth.

Those skilled in the art will appreciate that the present invention is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

One skilled in the art will recognize many methods and materials similar or equivalent to those described herein, which could be used in the practice of the present invention. The present invention is in no way limited to the methods and materials described.

All of the patents and publications referred to herein are incorporated by reference in their entirety.

The present invention is not to be limited in scope by the specific examples described herein, which are intended for the purpose of exemplification only. Functionally-equivalent products, compositions and methods are clearly within the scope of the present invention.

Any example or embodiment of the present invention herein shall be taken to apply mutatis mutandis to any other example or embodiment of the invention unless specifically stated otherwise.

Unless specifically defined otherwise, all technical and scientific terms used herein shall be taken to have the same meaning as commonly understood by one of ordinary skill in the art (for example, in diagnostic technology, radioimaging, cell culture, molecular genetics, immunology, immunohistochemistry, protein chemistry, and biochemistry).

The term “and/or”, e.g., “X and/or Y” shall be understood to mean either “X and Y” or “X or Y” and shall be taken to provide explicit support for both meanings or for either meaning.

### Selected Definitions

The term “isolated protein” or “isolated polypeptide” is a protein or polypeptide that by virtue of its origin or source of derivation is not associated with naturally-associated components that accompany it in its native state; is substantially free of other proteins from the same source. A protein may be rendered substantially free of naturally associated components or substantially purified by isolation, using protein purification techniques known in the art. By “substantially purified” is meant the protein is substantially free of contaminating agents, e.g., at least about 70% or 75% or 80% or 85% or 90% or 95% or 96% or 97% or 98% or 99% free of contaminating agents.

The term “recombinant” shall be understood to mean the product of artificial genetic recombination. Accordingly, in the context of a recombinant protein comprising an antibody antigen binding domain, this term does not encompass an antibody naturally-occurring within a subject's body that is the product of natural recombination that occurs during B cell maturation. However, if such an antibody is isolated, it is to be considered an isolated protein comprising an antibody antigen binding domain. Similarly, if nucleic acid encoding the protein is isolated and expressed using recombinant means, the resulting protein is a recombinant protein comprising an antibody antigen binding domain. A recombinant protein also encompasses a protein expressed by artificial recombinant means when it is within a cell, tissue or subject, e.g., in which it is expressed.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulphide bond. Examples of non-covalent bonds include hydrogen bonds, ionic bonds, Van der Waals forces, and hydrophobic interactions.

The term “polypeptide” or “polypeptide chain” will be understood from the foregoing paragraph to mean a series of contiguous amino acids linked by peptide bonds.

As used herein, the term “antigen binding protein” is used interchangeably with “antigen binding domain” and shall be taken to mean a region of an antibody that is capable of specifically binding to an antigen, i.e., a VH or a VL or an Fv comprising both a VH and a VL. The antigen binding domain need not be in the context of an entire antibody, e.g., it can be in isolation (e.g., a domain antibody) or in another form, e.g., as described herein, such as a scFv.

For the purposes for the present disclosure, the term “antibody” includes a protein capable of specifically binding to one or a few closely related antigens by virtue of an antigen binding domain contained within a Fv. This term includes four chain antibodies (e.g., two light chains and two heavy chains), recombinant or modified antibodies (e.g., chimeric antibodies, humanized antibodies, human antibodies, CDR-grafted antibodies, primatized antibodies, de-immunized antibodies, synhumanized antibodies, half-antibodies, bispecific antibodies). An antibody generally comprises constant domains, which can be arranged into a constant region or constant fragment or fragment crystallizable (Fc). Exemplary forms of antibodies comprise a four-chain structure as their basic unit. Full-length antibodies comprise two heavy chains (~50 to 70 kD) covalently linked and two light chains (~23 kDa each). A light chain generally comprises a variable region (if present) and a constant domain and in mammals is either a κ light chain or a λ light chain. A heavy chain generally comprises a variable region and one or two constant domain(s) linked by a hinge region to additional constant domain(s). Heavy chains of mammals are of one of the following types α, δ, ε, γ, or μ. Each light chain is also covalently linked to one of the heavy chains. For example, the two heavy chains and the heavy and light chains are held together by inter-chain disulfide bonds and by non-covalent interactions. The number of inter-chain disulfide bonds can vary among different types of antibodies. Each chain has an N-terminal variable region (VH or VL wherein each are ~110 amino acids in length) and one or more constant domains at the C-terminus. The constant domain of the light chain (CL which is ~110 amino acids in length) is aligned with and disulfide bonded to the first constant domain of the heavy chain (CH1 which is 330 to 440 amino acids in length). The light chain variable region is aligned with the variable region of the heavy chain. The antibody heavy chain can comprise 2 or more additional CH domains (such as, CH2, CH3 and the like) and can comprise a hinge region between the CH1 and CH2 constant domains. Antibodies can be of any type (e.g., IgG, IgE, IgM, IgD, IgA, and IgY), class (e.g., IgG1, IgG2, IgG3, IgG4, IgA1 and IgA2) or subclass. In one example, the antibody is a murine (mouse or rat) antibody or a primate (such as, human) antibody. In one example the antibody heavy chain is missing a C-terminal lysine residue. In one example, the antibody is humanized, synhumanized, chimeric, CDR-grafted or deimmunized.

The terms “full-length antibody”, “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof.

As used herein, “variable region” refers to the portions of the light and/or heavy chains of an antibody as defined herein that is capable of specifically binding to an antigen and, includes amino acid sequences of complementarity determining regions (CDRs); i.e., CDR1, CDR2, and CDR3, and framework regions (FRs). For example, the variable region comprises three or four FRs (e.g., FR1, FR2, FR3 and optionally FR4) together with three CDRs. VH refers to the variable region of the heavy chain. VL refers to the variable region of the light chain.

As used herein, the term “subject” shall be taken to mean any animal including humans, for example a mammal. Exemplary subjects include but are not limited to humans and non-human primates. For example, the subject is a human.

“Antibodies” or “immunoglobulins” or “Igs” are gamma globulin proteins that are found in blood, or other bodily fluids of vertebrates that function in the immune system to bind antigen, hence identifying and neutralizing foreign objects.

Antibodies are generally a heterotetrameric glycoprotein composed of two identical light (L) chains and two identical heavy (H) chains. Each L chain is linked to a H chain by one covalent disulfide bond. The two H chains are linked to each other by one or more disulfide bonds depending on the H chain isotype. Each H and L chain also has regularly spaced intrachain disulfide bridges.

H and L chains define specific Ig domains. More particularly, each H chain has at the N-terminus, a variable domain (VH) followed by three constant domains (CH) for each of the α and γ chains and four CH domains for μ and ε isotypes. Each L chain has at the N-terminus, a variable domain (V L) followed by a constant domain (CL) at its other end. The VL is aligned with the VH and the CL is aligned with the first constant domain of the heavy chain (CH1).

Antibodies can be assigned to different classes or isotypes. There are five classes of immunoglobulins: IgA, IgD, IgE, IgG, and IgM, having heavy chains designated α, δ, ϵ, γ, and μ, respectively. The γ and α classes are further divided into subclasses on the basis of relatively minor differences in CH sequence and function, e.g., humans express the following subclasses: IgG1, IgG2, IgG3, IgG4, IgA1, and IgA2. The L chain from any vertebrate species can be assigned to one of two clearly distinct types, called kappa and lambda, based on the amino acid sequences of their constant domains.

The constant domain includes the Fc portion which comprises the carboxy-terminal portions of both H chains held together by disulfides. The effector functions of antibodies such as ADCC are determined by sequences in the Fc region, which region is also the part recognized by Fc receptors (FcR) found on certain types of cells.

The pairing of a VH and VL together forms a “variable region” or “variable domain” including the amino-terminal domains of the heavy or light chain of the antibody. The variable domain of the heavy chain may be referred to as “VH.” The variable domain of the light chain may be referred to as “VL.” The V domain contains an antigen binding protein which affects antigen binding and defines specificity of a particular antibody for its particular antigen. V regions span about 110 amino acid residues and consist of relatively invariant stretches called framework regions (FRs) (generally about 4) of 15-30 amino acids separated by shorter regions of extreme variability called “hypervariable regions” (generally about 3) that are each 9-12 amino acids long. The FRs largely adopt a β-sheet configuration and the hypervariable regions form loops connecting, and in some cases forming part of, the 3-sheet structure.

“Hypervariable region”, “HVR”, or “HV” refers to the regions of an antibody variable domain which are hypervariable in sequence and/or form structurally defined loops. Generally, antibodies comprise six hypervariable regions; three in the VH (H1, H2, H3), and three in the VL (L1, L2, L3). A number of hypervariable region delineations are in use and are encompassed herein.

As used herein, the term “complementarity determining regions” (syn. CDRs; i.e., CDR1, CDR2, and CDR3) refers to the amino acid residues of an antibody variable region the presence of which are major contributors to specific antigen binding. Each variable region domain (VH or VL) typically has three CDRs identified as CDR1, CDR2 and CDR3. The CDRs of VH are also referred to herein as CDR H1, CDR H2 and CDR H3, respectively, wherein CDR H1 corresponds to CDR 1 of VH, CDR H2 corresponds to CDR 2 of VH and CDR H3 corresponds to CDR 3 of VH. Likewise, the CDRs of VL are referred to herein as CDR L1, CDR L2 and CDR L3, respectively, wherein CDR L1 corresponds to CDR 1 of VL, CDR L2 corresponds to CDR 2 of VL and CDR L3 corresponds to CDR 3 of VL. In one example, the amino acid positions assigned to CDRs and FRs are defined according to Kabat Sequences of Proteins of Immunological Interest, National Institutes of Health, Bethesda, Md., 1987 and 1991 (also referred to herein as “the Kabat numbering system”). In another example, the amino acid positions assigned to CDRs and FRs are defined according to the Enhanced Chothia Numbering Scheme (http://www.bioinfo.org.uk/mdex.html). The present invention is not limited to FRs and CDRs as defined by the Kabat numbering system, but includes all numbering systems, including the canonical numbering system or of Chothia and Lesk J. Mol. Biol. 196: 901-917, 1987; Chothia et al., Nature 342: 877-883, 1989; and/or AI-Lazikani et al., J. Mol. Biol. 273: 927-948, 1997; the numbering system of Honnegher and Plükthun J. Mol. Biol. 309: 657-670, 2001; or the IMGT system discussed in Giudicelli et al., Nucleic Acids Res. 25: 206-211 1997. In one example, the CDRs are defined according to the Kabat numbering system. Optionally, heavy chain CDR2 according to the Kabat numbering system does not comprise the five C-terminal amino acids listed herein or any one or more of those amino acids are substituted with another naturally-occurring amino acid. In this regard, Padlan et al., FASEB J., 9: 133-139, 1995 established that the five C-terminal amino acids of heavy chain CDR2 are not generally involved in antigen binding.

“Framework” or “FR” residues are those variable domain residues other than the hypervariable region or CDR residues herein defined. The FRs of VH are also referred to herein as FR H1, FR H2, FR H3 and FR H4, respectively, wherein FR H1 corresponds to FR 1 of VH, FR H2 corresponds to FR 2 of VH, FR H3 corresponds to FR 3 of VH and FR H4 corresponds to FR 4 of VH. Likewise, the FRs of VL are referred to herein as FR L1, FR L2, FR L3 and FR L4, respectively, wherein FR L1 corresponds to FR 1 of VL, FR L2 corresponds to FR 2 of VL, FR L3 corresponds to FR 3 of VL and FR L4 corresponds to FR 4 of VL.

“A peptide for forming an antigen binding protein” generally refers to a peptide that may form a conformation that confers the specificity of an antibody for antigen. Examples include whole antibody or whole antibody related structures, whole antibody fragments including a variable domain, variable domains and fragments thereof, including light and heavy chains, or fragments of light and heavy chains that include some but not all of hypervariable regions or constant regions.

An “intact” or “whole” antibody is one which comprises an antigen-binding protein as well as a CL and at least heavy chain constant domains, CH1, CH2 and CH3. The constant domains may be native sequence constant domains (e.g. human native sequence constant domains) or amino acid sequence variant thereof.

“Whole antibody related structures” include multimerized forms of whole antibody.

“Whole antibody fragments including a variable domain” include Fab, Fab′, F(ab′)2, and Fv fragments; diabodies; linear antibodies, single-chain antibody molecules; and multispecific antibodies formed from antibody fragments.

The Fab fragment consists of an entire L chain along with the variable region domain of the H chain (VH), and the first constant domain of one heavy chain (CHI). Each Fab fragment is monovalent with respect to antigen binding, i.e., it has a single antigen-binding protein.

A Fab′ fragment differs from Fab fragments by having additional few residues at the carboxy terminus of the CHI domain including one or more cysteines from the antibody hinge region. Fab′-SH is the designation herein for Fab′ in which the cysteine residue(s) of the constant domains bear a free thiol group.

A F(ab′)2 fragment roughly corresponds to two disulfide linked Fab fragments having divalent antigen-binding activity and is still capable of cross-linking antigen.

An “Fv” is an antibody fragment which contains a complete antigen-recognition and—binding site. This fragment consists of a dimer of one heavy- and one light-chain variable region domain in tight, non-covalent association.

In a single-chain Fv (scFv) species, one heavy- and one light-chain variable domain can be covalently linked by a flexible peptide linker such that the light and heavy chains can associate in a “dimeric” structure analogous to that in a two-chain Fv species. From the folding of these two domains emanate six hypervariable loops (3 loops each from the H and L chain) that contribute the amino acid residues for antigen binding and confer antigen binding specificity to the antibody.

“Single-chain Fv” also abbreviated as “sFv” or “scFv” are antibody fragments that comprise the VH and VL antibody domains connected to form a single polypeptide chain. Preferably, the scFv polypeptide further comprises a polypeptide linker between the VH and VL domains which enables the scFv to form the desired structure for antigen binding.

A “single variable domain” is half of an Fv (comprising only three CDRs specific for an antigen) that has the ability to recognize and bind antigen, although at a lower affinity than the entire binding site.

“Diabodies” refers to antibody fragments with two antigen-binding sites, which fragments comprise a heavy-chain variable domain (VH) connected to a light-chain variable domain (VL) in the same polypeptide chain (VH-VL). The small antibody fragments are prepared by constructing sFv fragments (see preceding paragraph) with short linkers (about 5-10 residues) between the VH and VL domains such that interchain but not intra-chain pairing of the V domains is achieved, resulting in a bivalent fragment, i.e., fragment having two antigen-binding sites.

Diabodies may be bivalent or bispecific. Bispecific diabodies are heterodimers of two “crossover” sFv fragments in which the VH and VL domains of the two antibodies are present on different polypeptide chains. Triabodies and tetrabodies are also generally known in the art.

An “isolated antibody” is one which has been identified and separated and/or recovered from a component of its pre-existing environment. Contaminant components are materials that would interfere with therapeutic uses for the antibody, and may include enzymes, hormones, and other proteinaceous or nonproteinaceous solutes.

A “human antibody” refers to an antibody which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Human antibodies can be prepared by administering the antigen to a transgenic animal that has been modified to produce such antibodies in response to antigenic challenge, but whose endogenous loci have been disabled.

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin.

“Monoclonal antibody” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site or determinant on the antigen. In addition to their specificity, the monoclonal antibodies are advantageous in that they may be synthesized uncontaminated by other antibodies. Monoclonal antibodies may be prepared by the hybridoma methodology, or may be made using recombinant DNA methods in bacterial, eukaryotic animal or plant cells. The “monoclonal antibodies” may also be isolated from phage antibody libraries.

The monoclonal antibodies herein include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. Chimeric antibodies of interest herein include “primatized” antibodies comprising variable domain antigen-binding sequences derived from a non-human primate (e.g. Old World Monkey, Ape etc.), and human constant region sequences.

“Binding affinity” generally refers to the strength of the sum total of noncovalent interactions between a single binding site of a molecule (e.g., an antibody) and its binding partner (e.g., an antigen). Generally, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention.

As used herein, the term “binds” in reference to the interaction of an antigen binding protein or an antigen binding domain thereof with an antigen means that the interaction is dependent upon the presence of a particular structure (e.g., an antigenic determinant or epitope) on the antigen. For example, an antibody recognizes and binds to a specific protein structure rather than to proteins generally. If an antibody binds to epitope “A”, the presence of a molecule containing epitope “A” (or free, unlabelled “A”), in a reaction containing labeled “A” and the protein, will reduce the amount of labelled “A” bound to the antibody.

As used herein, the term “specifically binds” or “binds specifically” shall be taken to mean that an antigen binding protein of the invention reacts or associates more frequently, more rapidly, with greater duration and/or with greater affinity with a particular antigen or cell expressing same than it does with alternative antigens or cells.

As used herein, the term “does not detectably bind” shall be understood to mean that an antigen binding protein, e.g., an antibody, binds to a candidate antigen at a level less than 10%, or 8% or 6% or 5% above background. The background can be the level of binding signal detected in the absence of the protein and/or in the presence of a negative control protein (e.g., an isotype control antibody) and/or the level of binding detected in the presence of a negative control antigen. The level of binding is detected using biosensor analysis (e.g. Biacore) in which the antigen binding protein is immobilized and contacted with an antigen.

As used herein, the term “does not significantly bind” shall be understood to mean that the level of binding of an antigen binding protein of the invention to a polypeptide is not statistically significantly higher than background, e.g., the level of binding signal detected in the absence of the antigen binding protein and/or in the presence of a negative control protein (e.g., an isotype control antibody) and/or the level of binding detected in the presence of a negative control polypeptide. The level of binding is detected using biosensor analysis (e.g. Biacore) in which the antigen binding protein is immobilized and contacted with an antigen.

An “affinity matured” antibody is one with one or more alterations in one or more HVRs thereof which result in an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art.

“ADCC” refers to a process called antibody-dependent cellular cytotoxicity, which is an immune response mediated primarily by natural killer (NK) cells in humans. In ADCC, FcγRIII on the surface of an NK cell recognizes the Fe region of antibody that is bound to antigen displayed on the surface of a target cell. This activates the NK cell, which releases perforins and granzymes, leading to lysis and apoptosis of the target cells.

“CDC” refers to a complex process called complement-dependent cytotoxicity that can lead to cell killing through the action of a cascade of proteins that can act through either of two major pathways.

“ADCP” refers to a process called antibody dependent cell-mediated phagocytosis. In this Fe receptor-mediated process, target cells to which antibodies are bound are engulfed by phagocytic cells, such as macrophage, monocytes, neutrophils, and dendritic cells. Multiple Fc receptors are involved in this process.

A “blocking” antibody or an “antagonist” antibody is one which inhibits or reduces biological activity of the antigen it binds. Preferred blocking antibodies or antagonist antibodies substantially or completely inhibit the biological activity of the antigen.

An “agonist antibody”, as used herein, is an antibody which mimics at least one of the functional activities of a polypeptide of interest.

As meant herein, an “Fc region” is a dimer consisting of two polypeptide chains joined by one or more disulfide bonds, each chain comprising part or all of a hinge domain plus a CH2 and a CH3 domain. Each of the polypeptide chains is referred to as an “Fc polypeptide chain.” To distinguish the two Fe polypeptide chains, one is referred to herein as an “A chain” and the other is referred to as a “B chain.” More specifically, the Fc regions contemplated for use with the present invention are IgG Fc regions, which can be mammalian or human IgG1, IgG2, IgG3, or IgG4 Fc regions. Among human IgG1 Fc regions, at least two allelic types are known.

An “Fc-containing protein,” as meant herein, is a protein comprising an Fc region as described herein and a binding region that binds to a target molecule. The term “Fc containing protein” encompasses an antibody or an Fc fusion protein that contains an Fc region.

The words “treat” or “treatment” refer to therapeutic treatment wherein the object is to slow down (lessen) an undesired physiological change or disorder. For the purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. Treatment can also mean prolonging survival as compared to expected survival if not receiving treatment. Treatment may not necessarily result in the complete clearance of a disease or disorder but may reduce or minimise complications and side effects of infection and the progression of a disease or disorder.

The phrase “pharmaceutically acceptable” indicates that the substance or composition must be compatible chemically and/or toxicologically, with the other ingredients comprising a formulation, and/or the mammal being treated therewith.

### Antibodies

The present invention relates to the use of radiolabelled olaratumab or antigen binding fragments thereof, for the detection, imaging, diagnosis of cancers and also for the selection, classification and stratification of cancers and cancer patients for treatment with an inhibitor of PDGFRα (wherein in certain embodiments, the PDGFRα may be olaratumab or an antigen binding fragment or derivative thereof). As used herein, olaratumab may also be referred to as IMC-3G3 and is an antibody that specifically binds to the human PDGFRα.

As used herein, the term “specifically binds” or “binds specifically” shall be taken to mean that an agent for use according to the invention, reacts or associates more frequently, more rapidly, with greater duration and/or with greater affinity with a PDGFRalpha or cell expressing same than it does with alternative antigens or cells. For example, an antigen binding protein that binds to PDGFRalpha with materially greater affinity (e.g., 1.5 fold or 2 fold or 5 fold or 10 fold or 20 fold or 40 fold or 60 fold or 80 fold to 100 fold or 150 fold or 200 fold) than it does to other antigens.

Methods for assessing binding to a protein (e.g. PDGFRalpha) are known in the art, e.g., as described in Scopes (In: Protein purification: principles and practice, Third Edition, Springer Verlag, 1994). Such a method generally involves immobilizing the agent (e.g. antibody) and contacting it with labeled target (in the case of an antibody, the antigen). Following washing to remove non-specific bound protein, the amount of label and, as a consequence, bound antigen is detected. Of course, the antigen binding site can be labeled and the antigen immobilized. Panning-type assays can also be used. Alternatively, or additionally, surface plasmon resonance assays can be used.

### Constant Regions

Any antibody and/or antigen binding fragment thereof as described herein for use in the present invention may comprise a constant region of an antibody. This includes antigen binding fragments of an antibody fused to an Fc.

Sequences of constant regions useful for producing the antibodies or antigen binding fragment thereof as described herein may be obtained from a number of different sources. In some examples, the constant region or portion thereof of the protein is derived from a human antibody. The constant region or portion thereof may be derived from any antibody class, including IgM, IgG, IgD, IgA and IgE, and any antibody isotype, including IgG1, IgG2, IgG3 and IgG4. In one example, the constant region is human isotype IgG4 or a stabilized IgG4 constant region.

In various embodiments of the invention, the Fc region of the antibody may comprise one or more substitutions for altering effector function (including increasing or decreasing effector functions), and circulation half-life. Various examples of such substitutions and modifications are described in Saunders (2019) Front. Immunol. article 1296, incorporated herein by reference in its entirety.

In one example, the Fc region of the constant region has a reduced ability to induce effector function, e.g., compared to a native or wild-type human IgG1 or IgG3 Fc region. In one example, the effector function is antibody-dependent cell-mediated cytotoxicity (ADCC) and/or antibody-dependent cell-mediated phagocytosis (ADCP) and/or complement-dependent cytotoxicity (CDC). Methods for assessing the level of effector function of an Fc region containing protein are known in the art and/or described herein.

In one example, the Fc region is an IgG4 Fc region (i.e., from an IgG4 constant region), e.g., a human IgG4 Fc region. Sequences of suitable IgG4 Fc regions will be apparent to the skilled person and/or available in publically available databases (e.g., available from National Center for Biotechnology Information).

In one example, the constant region is a stabilized IgG4 constant region. The term “stabilized IgG4 constant region” will be understood to mean an IgG4 constant region that has been modified to reduce Fab arm exchange or the propensity to undergo Fab arm exchange or formation of a half-antibody or a propensity to form a half antibody. “Fab arm exchange” refers to a type of protein modification for human IgG4, in which an IgG4 heavy chain and attached light chain (half-molecule) is swapped for a heavy-light chain pair from another IgG4 molecule. Thus, IgG4 molecules may acquire two distinct Fab arms recognizing two distinct antigens (resulting in bispecific molecules). Fab arm exchange occurs naturally in vivo and can be induced in vitro by purified blood cells or reducing agents such as reduced glutathione. A “half antibody” forms when an IgG4 antibody dissociates to form two molecules each containing a single heavy chain and a single light chain.

In one example, a stabilized IgG4 constant region comprises a proline at position 241 of the hinge region according to the system of Kabat (Kabat et al., Sequences of Proteins of Immunological Interest Washington DC United States Department of Health and Human Services, 1987 and/or 1991). This position corresponds to position 228 of the hinge region according to the EU numbering system (Kabat et al., Sequences of Proteins of Immunological Interest Washington DC United States Department of Health and Human Services, 2001 and Edelman et al., Proc. Natl. Acad. USA, 63, 78-85, 1969). In human IgG4, this residue is generally a serine. Following substitution of the serine for proline, the IgG4 hinge region comprises a sequence CPPC. In this regard, the skilled person will be aware that the “hinge region” is a proline-rich portion of an antibody heavy chain constant region that links the Fc and Fab regions that confers mobility on the two Fab arms of an antibody. The hinge region includes cysteine residues which are involved in inter-heavy chain disulfide bonds. It is generally defined as stretching from Glu226 to Pro243 of human IgG1 according to the numbering system of Kabat. Hinge regions of other IgG isotypes may be aligned with the IgG1 sequence by placing the first and last cysteine residues forming inter-heavy chain disulphide (S—S) bonds in the same positions (see for example WO2010/080538).

Additional examples of stabilized IgG4 antibodies are antibodies in which arginine at position 409 in a heavy chain constant region of human IgG4 (according to the EU numbering system) is substituted with lysine, threonine, methionine, or leucine (e.g., as described in WO2006/033386). The Fc region of the constant region may additionally or alternatively comprise a residue selected from the group consisting of: alanine, valine, glycine, isoleucine and leucine at the position corresponding to 405 (according to the EU numbering system). Optionally, the hinge region comprises a proline at position 241 (i.e., a CPPC sequence) (as described above).

In another example, the Fc region is a region modified to have reduced effector function, i.e., a “non-immunostimulatory Fc region”. For example, the Fc region is an IgG1 Fc region comprising a substitution at one or more positions selected from the group consisting of 268, 309, 330 and 331. In another example, the Fc region is an IgG1 Fc region comprising one or more of the following changes E233P, L234V, L235A and deletion of G236 and/or one or more of the following changes A327G, A330S and P331S (Armour et al., Eur J Immunol. 29:2613-2624, 1999; Shields et al., J Biol Chem. 276(9):6591-604, 2001). Additional examples of non-immunostimulatory Fc regions are described, for example, in Dall'Acqua et al., J Immunol. 177: 1129-1138 2006; and/or Hezareh J Virol; 75: 12161-12168, 2001).

Antibodies with reduced effector function include those with substitution of one or more of Fc region residues 238, 265, 269, 270, 297, 327 and 329 (as described in U.S. Pat. No. 6,737,056, incorporated herein by reference). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581). For example, an antibody variant may comprise an Fc region with one or more amino acid substitutions which diminish FcγR binding, e.g., substitutions at positions 234 and 235 of the Fc region (EU numbering of residues). For example, the substitutions are L234A and L235A (LALA) (See, e.g., WO 2012/130831). The substitutions may additionally include substitution of the proline residue at position 329, such as a P329G mutation to disable binding to FcR. Further, alterations may be made in the Fc region that result in altered (i.e., diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

In some aspects, the Fc region includes mutations to the complement (C1q) and/or to Fc gamma receptor (FcγR) binding sites. In some aspects, such mutations can render the antibody incapable of antibody directed cytotoxicity (ADCC) and complement directed cytotoxicity (CDC). One example of a CDC-deficient antibody is one that comprises a substitution at one or more of Glu318, Lys320, Pro 329, Pro331 and Lys322 (for example, K322A), wherein the numbering of the residues in the Fc region is according to the EU index as described in Kabat et al.

In another example, the Fc region is a chimeric Fc region, e.g., comprising at least one CH2 domain from an IgG4 antibody and at least one CH3 domain from an IgG1 antibody, wherein the Fc region comprises a substitution at one or more amino acid positions selected from the group consisting of 240, 262, 264, 266, 297, 299, 307, 309, 323, 399, 409 and 427 (EU numbering) (e.g., as described in WO2010/085682). Exemplary substitutions include 240F, 262L, 264T, 266F, 2970, 299A, 299K, 307P, 309K, 309M, 309P, 323F, 399S, and 427F.

### Additional Modifications

The present invention also contemplates additional modifications to an antibody or antigen binding protein comprising an Fc region or constant region.

The neonatal Fc-receptor (FcRn) is important for the metabolic fate of antibodies of the IgG class in vivo. The FcRn functions to salvage IgG from the lysosomal degradation pathway, resulting in reduced clearance and increased half-life. FcRn binds with high affinity to the CH2-CH3 portion of the Fc-region of an antibody of the class IgG. The interaction between an antibody of the class IgG and the FcRn is pH dependent and occurs in a 1:2 stoichiometry, i.e. one IgG antibody molecule can interact with two FcRn molecules via its two heavy chain Fc-region polypeptides (see e.g. Huber, A. H., et al, J. Mol. Biol. 230 (1993) 1077-1083).

In certain embodiments of the invention, an antibody may comprise one or more amino acid substitutions that increase the half-life of the protein. For example, the antibody comprises a Fc region comprising one or more amino acid substitutions that increase the affinity of the Fc region for the neonatal Fc region (FcRn). For example, the Fc region has increased affinity for FcRn at lower pH, e.g., about pH 6.0, to facilitate Fc/FcRn binding in an endosome. In one example, the Fc region has increased affinity for FcRn at about pH 6 compared to its affinity at about pH 7.4, which facilitates the re-release of Fc into blood following cellular recycling. These amino acid substitutions are useful for extending the half-life of a protein, by reducing clearance from the blood.

Exemplary amino acid substitutions include T250Q and/or M428L or T252A, T254S and T266F or M252Y, S254T and T256E or H433K and N434F according to the EU numbering system. Additional or alternative amino acid substitutions are described, for example, in US20070135620 or U.S. Pat. No. 7,083,784.

In further embodiments, the antibody comprises one or more amino acid substitutions that decrease the half-life of the protein. For example, the antibody comprises a Fc region comprising one or more amino acid substitutions that decrease or reduce the affinity of the Fc region for the neonatal Fc region (FcRn).

The present invention therefore provides for an antibody having substitutions in the CH2 and/or CH3 domains of the constant region and comprising substitutions at one or more of residues His310, His435, Tyr436 and Ile253 (Kabat EU index numbering), thereby altering FcRn binding affinity and/or serum half-life of said antibody relative to a naturally occurring antibody.

In some examples, the amino acid at position 310 and/or 435 of the antibody may be alanine, glutamic acid, aspartic acid, leucine, isoleucine, arginine, proline, glutamine, methionine, serine, threonine, lysine, asparagine, phenylalanine, tyrosine, tryptophan, cysteine, valine or glycine.

Preferably, the residue at position 310 is selected from alanine, or glutamic acid or glutamine; or amino acid residue 435 from the heavy chain constant region is selected from arginine, glutamine or alanine. In other preferred embodiments, the antibody has an alanine residue at position 310 and glutamine residue at position 435.

In a preferred embodiment of the present invention, the binding affinity for FcRn and/or the serum half-life of the modified antibody is decreased by at least about 2-fold, 3-fold, 4-fold, 5-fold, 10-fold, 15-fold, 20-fold, 25-fold, 30-fold, 40-fold, 50-fold, 60-fold, 70-fold, 80-fold, 90-fold, or 100-fold. In a preferred embodiment of the present invention, the binding affinity for FcRn and/or the serum half-life of said modified antibody is reduced by at least about 20%, 30%, 40%, 50%, 60%, 70%, 80%, 85%, 90%, 95%, 97%, 98%, or 99%.

The antibody may also comprise amino acid substitutions at residues equivalent to Ser228 and/or Leu235 of the constant heavy chain region, such as Ser228Pro and/or Leu235Glu.

### Protein Production

The production of an antigen binding protein of the invention generally requires an expression vector containing a polynucleotide that encodes the antigen binding protein of the invention. A polynucleotide encoding an antigen binding protein of the invention may be obtained and sub cloned into a vector for the production of an antigen binding protein by recombinant DNA technology using techniques well-known in the art, including techniques described herein. Many different expression systems are contemplated including the use of mammalian cells including human cells for production and secretion of antigen binding proteins. Examples of cells include 293F, CHO and the NSO cell line.

Expression vectors containing protein coding sequences and appropriate transcriptional and translational control signals can be constructed using methods known in the art. These include in vitro recombinant DNA techniques, synthetic techniques and in vivo genetic recombination. In certain embodiments there is provided a replicable vector having a nucleic acid encoding an antigen binding protein operably linked to a promoter.

Cells transfected with an expression vector may be cultured by conventional techniques to produce an antigen binding protein. Thus, in certain embodiments, there is provided host cells or cell transfectants containing a polynucleotide encoding an antigen binding protein of the invention operably linked to a promoter. The promoter may be heterologous. A variety of host-expression vector systems may be utilized and in certain systems the transcription machinery of the vector system is particularly matched to the host cell. For example, mammalian cells such as Chinese hamster ovary cells (CHO) may be transfected with a vector including the major intermediate early gene promoter element from human cytomegalovirus. Additionally or alternatively, a host cell may be used that modulates the expression of inserted sequences, or modifies and processes the gene product as required, including various forms of post translational modification. Examples of mammalian host cells having particular post translation modification processes include CHO, VERY, BHK, Hela, COS, MDCK, 293, 3T3, W138, BT483, Hs578T, HTB2, BT20 and T47D, NSO, CRL703O and HsS78Bst cells.

Depending upon the use intended for the protein molecule, a number of bacterial expression vectors may be advantageously selected. In one example, vectors that cause the expression of high levels of fusion protein products that are readily purified, such as the E. coli expression vector pUR278 may be used where a large quantity of an antigen binding protein is to be produced. The expression product may be produced in the form of a fusion protein with lacZ. Other bacterial vectors include pIN vectors and the like. pGEX vectors may also be used to express foreign polypeptides as fusion proteins with glutathione-S-transferase (GST). These fusion proteins are generally soluble and can easily be purified from lysed cells by adsorption and binding to glutathione-agarose affinity matrix followed by elution in the presence of free glutathione. A thrombin and/or factor Xa protease cleavage site may be provided in the expressed polypeptide so that the cloned target gene product can be released from the GST moiety.

Autographa californica nuclear polyhedrosis virus (AcNPV) may be used as a vector to express foreign genes in an insect system including Spodoptera frugiperda cells. The particular promoter used may depend on where the protein coding is inserted into the sequence. For example, the sequence may be cloned individually into the polyhedrin gene and placed under control of the polyhedrin promoter.

Virus based expression systems may be utilized with mammalian cells such as an adenovirus whereby the coding sequence of interest may be ligated to the adenoviral late promoter and tripartite leader sequence. In vitro or in vivo recombination may then be used to insert this chimeric gene into the adenoviral genome. Insertions into region E1 or E3 will result in a viable recombinant virus that is capable of expressing the antigen binding protein in infected host cells. Specific initiation signals including the ATG initiation codon and adjacent sequences may be required for efficient translation of inserted antigen binding protein coding sequences. Initiation and translational control signals and codons can be obtained from a variety of origins, both natural and synthetic. Transcription enhancer elements and transcription terminators may be used to enhance the efficiency of expression of a viral based system.

Where long-term, high-yield production of recombinant proteins is required, stable expression is preferred. Generally a selectable marker gene is used whereby following transfection, cells are grown for 1-2 days in an enriched media and then transferred to a medium containing a selective medium in which cells containing the corresponding selectable marker, for example, antibiotic resistance can be screened. The result is that cells that have stably integrated the plasmid into their chromosomes grow and form foci that in turn can be cloned and expanded into cell lines. The herpes simplex virus thymidine kinase, hypoxanthineguanine phosphoribosyltransferase and adenine phosphoribosyltransferase genes are examples of genes that can be employed in tk−, hgprt− or aprT− cells, respectively, thereby providing appropriate selection systems. The following genes: dhfr, which confers resistance to methotrexate; gpt, which confers resistance to mycophenolic acid; neo, which confers resistance to the aminoglycoside G-418; and hygro, which confers resistance to hygromycin are examples of genes that can be used in anti-metabolite selection systems.

An antigen binding protein of the invention may be purified by a recombinant expression system by known methods including ion exchange chromatography, affinity chromatography (especially affinity for the specific antigens Protein A or Protein G) and gel filtration column chromatography), centrifugation, differential solubility, or by any other standard technique for the purification of proteins. Purification may be facilitated or assisted by providing the antigen binding protein in the form of a fusion protein.

Large quantities of the antigen binding proteins of the invention may be produced by a scalable process starting with a pilot expression system in a research laboratory that is scaled up to an analytical scale bioreactor (typically from 5 L to about 50 L bioreactors) or production scale bioreactors (for example, but not limited to 75 L, 100 L, 150 L, 300 L, or 500 L). Desirable scalable processes include those wherein there are low to undetectable levels of aggregation as measured by HPSEC or rCGE, typically no more than 5% aggregation by weight of protein down to no more than 0.5% by weight aggregation of protein. Additionally or alternatively, undetectable levels of fragmentation measured in terms of the total peak area representing the intact antigen binding protein may be desired in a scalable process so that at least 80% and as much as 99.5% or higher of the total peak area represents intact antigen binding protein. In other embodiments, the scalable process of the invention produces antigen binding proteins at production efficiency of about from 10 mg/L to about 300 mg/L or higher.

Various techniques have been developed for the production of antibody fragments including proteolytic digestion of intact antibodies and recombinant expression in host cells. With regard to the latter, as described below, Fab, Fv and scFv antibody fragments can all be expressed in and secreted from E. coli, antibody fragments can be isolated from the antibody phage libraries and Fab′-SH fragments can be directly recovered from E. coli and chemically coupled to form F(ab′)2 fragments. In another approach, F(ab′)2 fragments are isolated directly from recombinant host cell culture.

In another embodiment there is provided a vector including a nucleic acid described above. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

The antigen binding site may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the antigen binding site-encoding DNA that is inserted into the vector. The signal sequence may be a prokaryotic signal sequence selected, for example, from the group of the alkaline phosphatase, penicillinase, Ipp, or heat-stable enterotoxin II leaders. For yeast secretion the signal sequence may be, e.g., the yeast invertase leader, alpha factor leader, or acid phosphatase leader or the C. albicans glucoamylase leader. In mammalian cell expression, mammalian signal sequences may be used to direct secretion of the protein, such as signal sequences from secreted polypeptides of the same or related species, as well as viral secretory leaders.

Polynucleotide sequences encoding polypeptide components of the antigen binding protein of the invention can be obtained using standard recombinant techniques as described above. Polynucleotides can be synthesized using nucleotide synthesizer or PCR techniques. Once obtained, sequences encoding the polypeptides are inserted into a recombinant vector capable of replicating and expressing heterologous polynucleotides in prokaryotic hosts. Many vectors that are available and known in the art can be used for the purpose of the present invention. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides.

In general, plasmid vectors containing replicon and control sequences which are derived from species compatible with the host cell are used in connection with these hosts. Both expression and cloning vectors contain a nucleic acid sequence that enables the vector to replicate in one or more selected host cells, as well as marking sequences which are capable of providing phenotypic selection in transformed cells. Such sequences are well known for a variety of bacteria, yeast, and viruses. The origin of replication from the plasmid pBR322, which contains genes encoding ampicillin (Amp) and tetracycline (Tet) resistance and thus provides easy means for identifying transformed cells, is suitable for most Gram-negative bacteria, the 2 μm plasmid origin is suitable for yeast, and various viral origins (SV40, polyoma, adenovirus, VSV or BPV) are useful for cloning vectors in mammalian cells. pBR322, its derivatives, or other microbial plasmids or bacteriophage may also contain, or be modified to contain, promoters which can be used by the microbial organism for expression of endogenous proteins.

In addition, phage vectors containing replicon and control sequences that are compatible with the host microorganism can be used as transforming vectors in connection with these hosts. For example, bacteriophage such as λGEM.TM.-11 may be utilized in making a recombinant vector which can be used to transform susceptible host cells such as E. coli LE392.

The expression vector of the invention may comprise two or more promoter-cistron (a cistron being segment of DNA that contains all the information for production of single polypeptide) pairs. A promoter is an untranslated regulatory sequence located upstream (5′) to a cistron that modulates its expression. Prokaryotic promoters typically fall into two classes, inducible and constitutive. Inducible promoter is a promoter that initiates increased levels of transcription of the cistron under its control in response to changes in the culture condition, e.g. the presence or absence of a nutrient or a change in temperature.

A large number of promoters recognized by a variety of potential host cells are well known. The selected promoter can be operably linked to cistron DNA encoding the light or heavy chain by removing the promoter from the source DNA via restriction enzyme digestion and inserting the isolated promoter sequence into the vector of the invention. Both the native promoter sequence and many heterologous promoters may be used to direct amplification and/or expression of the target genes. In some embodiments, heterologous promoters are utilized, as they generally permit greater transcription and higher yields of expressed target gene as compared to the native target polypeptide promoter.

Promoters recognized by a variety of potential host cells are well known. Promoters suitable for use with prokaryotic hosts include the PhoA promoter, the β-galactamase and lactose promoter systems, alkaline phosphatase, a tryptophan (trp) promoter system and hybrid promoters such as the tac or the trc promoter. Promoters for use in bacterial systems also will contain a Shine-Dalgarno (S.D.) sequence operably linked to the DNA encoding an antigen binding protein of the invention. However, other promoters that are functional in bacteria (such as other known bacterial or phage promoters) are suitable as well. Their nucleotide sequences have been published, thereby enabling a skilled person operably to ligate them to cistrons encoding the target light and heavy chains using linkers or adaptors to supply any required restriction sites.

In one aspect of the invention, each cistron within the recombinant vector comprises a secretion signal sequence component that directs translocation of the expressed polypeptides across a membrane. In general, the signal sequence may be a component of the vector, or it may be a part of the target polypeptide DNA that is inserted into the vector. The signal sequence selected for the purpose of this invention should be one that is recognized and processed (i.e. cleaved by a signal peptidase) by the host cell. For prokaryotic host cells that do not recognize and process the signal sequences native to the heterologous polypeptides, the signal sequence is substituted by a prokaryotic signal sequence selected, for example, from the group consisting of the alkaline phosphatase, penicillinase, Ipp, or heat-stable enterotoxin II (STII) leaders, LamB, PhoE, PelB, OmpA and MBP. In one embodiment of the invention, the signal sequences used in both cistrons of the expression system are STII signal sequences or variants thereof.

In another aspect, the production of the immunoglobulins according to the invention can occur in the cytoplasm of the host cell, and therefore does not require the presence of secretion signal sequences within each cistron. In that regard, immunoglobulin light and heavy chains are expressed, folded and assembled to form functional immunoglobulins within the cytoplasm. Certain host strains (e.g., the E. coli trxB strains) provide cytoplasm conditions that are favourable for disulfide bond formation, thereby permitting proper folding and assembly of expressed protein subunits.

The present invention provides an expression system in which the quantitative ratio of expressed polypeptide components can be modulated in order to maximize the yield of secreted and properly assembled antigen binding proteins of the invention. Such modulation is accomplished at least in part by simultaneously modulating translational strengths for the polypeptide components.

In terms of expression in eukaryotic host cells, the vector components generally include, but are not limited to, one or more of the following: a signal sequence, an origin of replication, one or more marker genes, an enhancer element, a promoter, and a transcription termination sequence.

A vector for use in a eukaryotic host cell may also contain a signal sequence or other polypeptide having a specific cleavage site at the N-terminus of the mature protein or polypeptide of interest. The heterologous signal sequence selected preferably is one that is recognized and processed {i.e., cleaved by a signal peptidase) by the host cell. In mammalian cell expression, mammalian signal sequences as well as viral secretory leaders, for example, the herpes simplex gD signal, are available.

The DNA for such precursor region is ligated in reading frame to DNA encoding the antibody.

Generally, an origin of replication component is not needed for mammalian expression vectors. For example, the SV40 origin may typically be used only because it contains the early promoter.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

An example of suitable selectable markers for mammalian cells are those that enable the identification of cells competent to take up the antigen binding protein-encoding nucleic acid, such as DHFR or thymidine kinase, metallothionein-I and -II, preferably primate metallothionein genes, adenosine deaminase, ornithine decarboxylase, etc. An appropriate host cell when wild-type DHFR is employed is the CHO cell line deficient in DHFR activity (e.g., ATCC CRL-9096), prepared and propagated. For example, cells transformed with the DHFR selection gene are first identified by culturing all of the transformants in a culture medium that contains methotrexate (Mtx), a competitive antagonist of DHFR. Alternatively, host cells (particularly wild-type hosts that contain endogenous DHFR) transformed or co-transformed with DNA sequences encoding an antibody, wild-type DHFR protein, and another selectable marker such as aminoglycoside 3 ′-phosphotransferase (APH) can be selected by cell growth in medium containing a selection agent for the selectable marker such as an aminoglycosidic antibiotic, e.g., kanamycin, neomycin, or G418.

Expression and cloning vectors usually contain a promoter operably linked to the antigen binding protein encoding nucleic acid sequence to direct mRNA synthesis. Promoters recognized by a variety of potential host cells are well known.

Eukaryotic genes generally have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Examples of suitable promoting sequences for use with yeast hosts include the promoters for 3-phosphoglycerate kinase or other glycolytic enzymes including enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization.

Antigen binding protein transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription of a DNA encoding the antigen binding protein by higher eukaryotes may be increased by inserting an enhancer sequence into the vector. Enhancer sequences include those known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding an antigen binding protein.

In another embodiment there is provided a cell including a vector or nucleic acid described above. The nucleic acid molecule or vector may be present in the genetically modified host cell or host either as an independent molecule outside the genome, preferably as a molecule which is capable of replication, or it may be stably integrated into the genome of the host cell or host.

The host cell of the present invention may be any prokaryotic or eukaryotic cell.

Examples of prokaryotic cells are those generally used for cloning like E. coli or Bacillus subtilis. Furthermore, eukaryotic cells comprise, for example, fungal or animal cells.

Examples for suitable fungal cells are yeast cells, preferably those of the genus Saccharomyces and most preferably those of the species Saccharomyces cerevisiae.

Examples of animal cells are, for instance, insect cells, vertebrate cells, preferably mammalian cells, such as e.g. HEK293, NSO, CHO, MDCK, U2-OS, Hela, NIH3T3, MOLT-4, Jurkat, PC-12, PC-3, IMR, NT2N, Sk-n-sh, CaSki, C33A. These host cells, e.g. CHO-cells, may provide post-translational modifications to the antibody molecules of the invention, including leader peptide removal, folding and assembly of H (heavy) and L (light) chains, glycosylation of the molecule at correct sides and secretion of the functional molecule.

Further suitable cell lines known in the art are obtainable from cell line depositories, like the American Type Culture Collection (ATCC).

In another embodiment there is provided an animal including a cell described above. In certain embodiments, animals and tissues thereof containing a transgene are useful in producing the antigen binding proteins of the invention. The introduction of the nucleic acid molecules as transgenes into non-human hosts and their subsequent expression may be employed for the production of the antigen binding proteins, for example, the expression of such a transgene in the milk of the transgenic animal provide for means of obtaining the antigen binding proteins in quantitative amounts. Useful transgenes in this respect comprise the nucleic acid molecules of the invention, for example, coding sequences for the antigen binding proteins described herein, operatively linked to promoter and/or enhancer structures from a mammary gland specific gene, like casein or beta-lactoglobulin. The animal may be non-human mammals, most preferably mice, rats, sheep, calves, dogs, monkeys or apes.

### Radiolabelled Antibodies

The skilled person will be familiar with standard methods for conjugating a detectable moiety such as a radionuclide (radiolabel) to an antibody or antigen binding fragment thereof.

As used herein, the term radionuclide may be used interchangeably with the term radioisotope and radiolabel.

In any embodiment of the invention, radiolabel may be conjugated to the olaratumab or antigen binding fragment thereof, directly via binding to single or multiple amino acid residues in the protein (e.g. halogenation of tyrosine residues) or indirectly (via a chelating agent or prosthetic group or linker) linked to the radioisotope (i.e. the radiolabel).

Examples of suitable chelating agents or linkers may be selected from the group consisting of: TMT (6,6″-bis[N,N″,N′″-tetra(carboxymethyl)aminomethyl)-4′-(3-amino-4-methoxyphenyl)-2,2′:6′,2″-terpyridine), DOTA (1, 4,7,10-tetraazacyclododecane-NN′,N″(N′″-tetraacetic acid, also known as tetraxetan), TCMC (the tetra-primary amide of DOTA), DO3A (1,4,7,10-Tetraazacyclododecane-1,4,7-tris(acetic acid)-10-(2-thioethyl)acetamide), CB-DO2A (4,10-bis(carboxymethyl)-1,4,7,10-tetraazabicyclo[5.5.2]tetradecan), NOTA (1,4,7-triazacyclononane-triacetic acid) Diamsar (3,6,10,13,16,19-hexaazabicyclo[6.6.6]eicosane-1,8-diamine), DTPA (Pentetic acid or diethylenetriaminepentaacetic acid), CHX-A″-DTPA ([(R)-2-Amino-3-(4-isothiocyanatophenyl)propyl]-trans-(S,S)-cyclohexane-1,2-diamine-pentaacetic acid), TETA (1,4,8,11-tetraazacyclotetradecane-1,4,8), 11-tetraacetic acid, Te2A (4,11-bis(carboxymethyl)-1,4,8,11-tetraazabicyclo[6.6.2]hexadecane), HBED, DFO (Desferrioxamine), DFOsq (DFO-squaramide) and HOPO (3,4,3-(LI-1,2-HOPO) or other chelating agent as described herein. Other known chelating moieties include 3p-C-NETA ({4-[2-(bis-carboxy-methylamino)-5-(4-nitrophenyl)pentyl]-7-carbo-xymethyl-[1,4,7]triazanonan-1-yl}acetic acid), 5p-C-NETA (2-({1-[4,7-bis(carboxymethyl)-1,4,7-triazanonan-1-yl]-7-(4-nitrophenyl)heptan-2-yl}(carbo-xymethyl) amino)acetic acid), NOTA (1,4,7-triazacyclononane-1,4,7-triacetic acid) and NODA (1,4,7-triazacyclononane-1,4-diacetic acid).

In certain embodiments, the olaratumab may be covalently coupled to the radioactive isotope ¹²⁴I. This isotope is a positron emitter that can be attached to antibodies e.g. as described by Larsson et al. (J. Nucl. Med. 33 (1992), 2020-2023) or U.S. Pat. No. 5,185,142, the content of which is herein incorporated by reference.

In any embodiment, radiolabelling of a protein or antibody is accomplished by covalent iodination, particularly with the Iodogen Reagent (1,3,4,6-tetrachloro-3a,6a-diphenyl glycoluril). Iodogen labeling is a solid phase oxidative method that is similar to the Chloramine-T method, but is generally considered to be milder, since the reaction takes place on the surface of the oxidant, minimizing exposure of the substrate (Salacinzki, P. R. P., et al., Anal. Biochem. 117:136 (1981)).

Chelators with radiometals and other halogenated radioisotopes may be bound to antibodies via one or more amino acid residues or reactive moieties in the protein/antibody, including but not limited to one or more lysine residues, tyrosine residues or thiol moieties.

In another example, the antibody may be conjugated to a bifunctional linker, for example, bromoacetyl, thiols, succinimide ester, TFP ester, a maleimide, or using any amine or thiol-modifying chemistry known in the art.

The skilled person will be familiar with standard methods for conjugating chelating agents to antibodies and derivatives or fragments thereof. In addition, the skilled person will be familiar with approaches for selecting a relevant chelating agent for pairing with a radiometal, for example as described in Chem. Soc. Rev., 2014,43, 260, incorporated herein by reference.

Examples of suitable radiolabels include: fluorine-18 (¹⁸F), gallium-67 and gallium-68 (⁶⁷Ga and ⁶⁸Ga), indium-111 (¹¹¹In), iodine-123 and iodine-124, technetium-99 (^{99m}Tc), terbium-155 and terbium-159 (¹⁵⁵Tb and ¹⁵⁹Tb) and zirconium-89 (⁸⁹Zr).

### Administration of Radiolabelled Olaratumab

The skilled person will appreciate that dosage of the agent for use in accordance with the methods of the invention will depend on various factors including the age, sex, height and weight of the subject to whom the agent is administered, and depending on the agent.

Preferably the olaratumab is administered to a subject or infused at a dose from about 1 mg to about 50 mg, preferably in a dose from about 5 mg to about 20 mg, and more preferably in a dose of about 10 mg. The specific activity of the radiolabeled antibody is preferably about 15 to about 20 MBq/mg, more preferably about 18 to about 19 MBq/mg.

In certain embodiments, the radiolabelled olaratumab is administered at a mass dose of about 5 to 20 mg of olaratumab by slow infusion.

The radiolabelled olaratumab antibody is usually administered as a pharmaceutical composition with a pharmaceutically acceptable carrier, e.g. physiological saline solution, optionally comprising a protein stabilizer such as human serum albumin (HSA). The radiolabelled olaratumab is preferably administered by infusion.

The radiolabelled olaratumab is preferably administered intravenously, preferably by infusion or intravenous injections. The administration of the antibody by infusion is preferably performed over a period of up to about 30 minutes, more preferably in about 15 minutes. Of course, the radiolabelled olaratumab can also be applied intraperitoneally or intramuscularly.

### Detection Methods

It will be appreciated that the methods for detecting or imaging of the radiolabelled olaratumab for use according to the invention will depend on the nature of the detectable moiety (radiolabel) of the antibody.

The step of detecting is preferably performed using PET, SPECT, or any other suitable method. Optionally, the detection method comprises PET/SPECT, PET/CT imaging or PET/MRI scanning.

Examples of in vivo methods for determining the presence or expression of PDGFRα in a tumour include use of in vivo/partial or whole body imaging techniques such as Positron Emission Tomography (PET) and single photon emission computed tomography (SPECT) imaging. Immuno-PET and immuno-SPECT imaging typically include the use of PDGFRα-binding molecules such as olaratumab that are conjugated to a radioisotope to enable non-invasive imaging of tissues and tumours expressing PDGFRα.

The in vivo detection step in the methods described above may be whole body imaging or local imaging at specific sites, such as but not limited to sites of anticipated or likely solid tumour growth.

In the case of SPECT, the radiolabelled olaratumab typically comprises a detectable agent in the form of a gamma-emitting radioisotope (radionuclide), normally through injection into the blood stream. Typically gamma-emitting radioisotopes for use in SPECT include ^{99m}Tc (technetium), ¹²³I or ¹³¹ I (iodine), indium-111 (¹¹¹In)⁶⁷Ga (gallium) and terbium-155 (¹⁵⁵Tb).

In any embodiment, the detection method may comprise Positron Emission Tomography (PET). In such instances, the radiolabelled olaratumab typically comprises a detectable agent in the form of a positron-emitting radioisotope (radionuclide), normally through injection into the blood stream. Examples of radioisotopes for use in PET include gallium-68 (⁶⁸Ga), zirconium-89 (⁸⁹Zr) and terbium-152 (¹⁵²Tb).

Following administration (preferably infusion) of the radiolabelled olaratumab, it may be practical to wait for a period of time to allow for the agent to accumulate at the site of cancer cells expressing tumours. Typically, the period of time will be at least about 2 days, at least about 3 days, at least about 4 days, at least about 5 days, at least about 6 days, at least about 7 days, at least about 8 days, at least about 9 days, at least about 10 days. Preferably, the time between administration of the agent and the detection of the agent (e.g. by PET or other method described here), the period is typically no more than about 10 days, or no more than about 15 days or no more than about 20 days.

In the case where the imaging or detection of the cancer is using PET, the PET imaging may be performed preferably within 7±2 days of infusion of the radiolabelled agent, in particular 5±2 days after the infusion, in order to obtain the optimal imaging results including accumulation of the agent at sites where PDGFRα is present.

### Cancers to be Detected, Imaged, Diagnosed or Classified

The present invention provides methods for identifying, detecting or imaging cancers in vivo and/or classifying such cancers for treatment. Such methods are expected to be useful in the diagnosis of cancers which express PDGFRalpha, especially soft tissue sarcomas, preferably without the need for additional, invasive techniques (such as biopsy collection and testing), for confirming diagnosis.

Accordingly in a preferred embodiment, the methods of the invention enable the diagnosis of any cancer recited herein, as the sole, principal or main mode of diagnosis of the cancer and preferably without the need for additional invasive methods of diagnosis including biopsy-related methods.

The methods of the invention are also expected to be useful for the staging of the progression of a cancer or the success of treatment of a cancer. Again, such methods provide the benefit of providing a non-invasive means for assessing the cancer in a subject.

In further embodiments, the methods are for identifying cancers which are likely sensitive to treatment with olaratumab or for selecting patients for subsequent treatment with an inhibitor of PDGFRα, such as olaratumab.

As used herein, the term “cancer” refers to a malignant growth or tumour resulting from an uncontrolled division of cells. The term “cancer” includes primary tumours and metastatic tumours.

A subject for whom the diagnosis or detecting or imaging of the cancer described herein, may be suspected of having or be at risk of having the cancer. A subject suspected of having a cancer may exhibit one or more symptoms of the cancer, may have a family history of the cancer or may have one or more genetic markers indicating a risk or likelihood of developing the cancer. A subject considered at risk of having the cancer may exhibit one or more symptoms of the cancer, may have a family history of the cancer or may have one or more genetic markers indicating a risk or likelihood of developing the cancer.

The PDGFRα-expressing cancer for which the diagnosis, detection or imaging thereof is required, classification of, selection of treatment thereof, or for which success of treatment is required to be determined, may be selected from the group consisting of: soft tissue sarcoma (STS), chondrosarcoma, leiomyosarcoma, liposarcoma, osteosarcoma, rhabdomyosarcoma, or any other cancer associated with expression of PDGFRα. Soft tissue sarcomas originate in such tissues as fat, muscles, nerves, tendons, and blood and lymph vessels.

Alternatively, the PDGFRα-expressing cancer for which the diagnosis, detection or imaging thereof is required, classification of, selection of treatment thereof, or for which success of treatment is required to be determined, may be selected from the group consisting of: gastrointestinal stromal tumour (GIST), non-small cell lung cancer (NSCLC), pancreatic cancer, hepatocellular carcinoma, breast cancer, neuroendocrine tumours, ovarian cancer, cervical cancer, uterine cancer (such as adenosquamous carcinoma of the uterine cervix), colorectal cancer, gastric cancer, salivary gland cancer, biliary cancer, glioma, prostate cancer and renal cell carcinoma.

The tumours can be hormone/androgen-dependent or hormone/androgen independent, and can have originated, for example, from prostate, breast, or lung.

Primary bone tumours to be classified or detected according to the invention include, but are not limited to, osteosarcomas, chondrosarcomas, fibrosarcomas, and hemangiosarcomas. Notably, malignant secondary (metastastic) tumours are far more common than primary bone tumours. Metastatic bone tumours to be classified or detected according to the invention can arise from a variety of sources, the most common of which are cancers of the prostate, breast, or lung. The source of a metastatic bone cancer will usually be apparent from a patients history. The tumours can be osteoblastic or osteolytic. Bone tumours that are PDGF-dependent can also be classified or detected according to the invention, as well as tumours that are “bone marrow” dependent.

Imaging or diagnosis of the cancer will typically be assessed following administration of the radiolabelled olaratumab, and detection thereof, by qualitatively assessing the detection of the radiolabelled olaratumab compared with conventional imaging. Quantitative assessment may be on a per lesion basis, including standardized uptake values (SUV) (SUVmax and SUVmean), SUV corrected for lean mass (SUL), metabolic tumor volume (MTV), and Tumour-to-background ratio (TBR).

The Tumor-to-Background Ratio (TBR) will typically be defined as the ratio of the lesion standardized uptake values (SUVmax) over the reference region SUV (liver, blood pool, etc.). Comparison between the number, size and other characteristics of lesions detected by PET scan and standard imaging modalities including high-resolution CT/MRI and other potential imaging per patient (depending on the tumor type), type of lesion and indication will be performed.

Imaging Qualitative visual analysis (presence or absence of localized agent uptake associated with tumor, as seen on contrast-enhanced CT, MRI or FDG PET/CT), may be used to evaluate concordance of tumor lesion detection between agent-specific PET/CT and conventional imaging. RECIST 1.1 criteria for conventional imaging may be used as the main tool for concordance comparison vs PET.

In addition to the above, all visible tumoral lesions at conventional imaging may also be compared to PET imaging result.

### Selection/Classification of Patients and Monitoring Response

According to the invention, patients for whom a cancer is detected according to the methods of the invention may be selected or classified for subsequent treatment with a PDGFRα inhibitor or antagonist. The present invention also provides methods for identifying cancers which are likely to be sensitive to treatment with a PDGFRα inhibitor. Thus, the invention also provides methods for identifying patients who would benefit from a treatment for a cancer that comprises an PDGFRα inhibitor, including associated methods of treatment.

As used herein, a cancer that is “sensitive” to treatment with PDGFRα inhibitor shall be understood to be a cancer that is inhibited (i.e., no longer proliferates) or is killed following administration of the PDGFRα inhibitor to the patient. It will be understood that sensitivity to treatment does not necessarily mean complete eradication of the cancer, but rather, a cessation or slowing of growth or proliferation of the tumour, prevention of metastasis, or even lysis/cell death of the tumour.

A PDGFRα antagonist can be an extracellular antagonist or an intracellular antagonist and more than one antagonist may be employed. Extracellular antagonists include, but are not limited to proteins or other biological molecules that bind to PDGFRα or one or more of its ligands (e.g., PDGF-AA, -AB, -BB, -CC). In an embodiment of the invention, an extracellular antagonist is inhibits binding of PDGFRα to its ligands. In one embodiment, the antagonist is an anti-PDGFRα antibody, such as, for example, IMC-3G3 (also known as olaratumab). In another embodiment, the binding protein is a soluble ligand binding fragment of PDGFRα. Intracellular IGF-IR antagonists can be biological molecules, but are usually small molecules. In one embodiment, the intracellular PDGFRα antagonist is AG1296. AG1296 (Calbiochem) is an inhibitor of PDGFβs, PDGFβs, and c-KIT, and also reacts with FIt3. Other small molecules that target PDGFRs include STI-571 (imatinib mesylate, Gleevec®, Novartis) and SU11248 (sunitinib malate, SUTENT®, Pfizer).

Preferably, the inhibitor of PDGFRα neutralizes PDGFRα and in certain embodiments may be an anti-PDGFRα antibody. Binding of a ligand, e.g., PDGF-AA, PDGF-AB, PDGF-BB or PDGF-CC, to an extracellular domain of PDGFRα stimulates receptor dimerization, autophosphorylation, activation of the receptor's internal, cytoplasmic tyrosine kinase domain, and initiation of multiple signal transduction and transactivation pathways involved in regulation of DNA synthesis (gene activation) and cell cycle progression or division. The anti-PDGFRα antibodies typically block ligand binding and/or receptor dimerization, and inhibit one or more of autophosphorylation, activation of tyrosine kinase activity and signal transduction. The anti-PDGFRα antibodies of the present invention can be specific for the extracellular ligand binding region of PDGFRα and prevent binding of a ligand of PDGFRα. Preferably, such anti-PDGFRα antibodies, or fragments thereof, bind PDGFRα at least as strongly as the natural ligands of PDGFRα. Alternatively or additionally, the antibodies can be specific for a region of the receptor monomer that would otherwise form a receptor dimer interface. Such antibodies block dimer formation, though ligand binding to a receptor monomer might or might not be blocked.

In one embodiment of the invention, the anti-PDGFRα antibodies reduce phosphorylation of PDGFRα by at least about 75%. In other embodiments, phosphorylation is reduced by at least about 85% or at least about 90%. In an embodiment of the invention, as a result of inhibition of PDGFRα signal transduction, phosphorylation or a downstream signal transduction pathway component (e.g., Akt, p42/p44, etc.) is reduced by at least about 40%, at least about 60%, or at least about 80%. Receptor neutralization can be determined using defined ligands (e.g., PDGF-AA, -AB, -BB, -CC), mixtures of such ligands, or preparations such as bone marrow aspirates that comprise PDGFs as well as other stimulatory growth factors.

Neutralization of PDGFRα includes inhibition, diminution, inactivation and/or disruption of one or more of these activities normally associated with signal transduction. Thus, neutralizing PDGFRα has various effects, including inhibition, diminution, inactivation and/or disruption of growth (proliferation and differentiation), angiogenesis (blood vessel recruitment, invasion, and metastasis), and cell motility and metastasis (cell adhesion and invasiveness).

Ex vivo assays, as described above, can also be utilized to determine PDGFRα neutralization. For example, human SKLMS-1 leiomyosarcoma cells (American Type Culture Collection (ATCC), Rockville, Md.; ATCC HTB-88™) or U118 glioblastoma cells (ATCC HTB-15™) stimulated with PDGF-AA can be used to assay PDGFRα inhibition. Growth inhibition can be ascertained using PDGFRα-expressing human tumour cells injected into a SCID mouse.

PDGFRα antagonists function by inhibiting signal transduction by PDGFRα expressed on the tumour cells themselves, or by inhibiting PDGFRα expressed on surrounding stromal cells that otherwise undergo paracrine stimulation by PDGFs expressed from tumour cells. Thus, antibodies such as EMC-3G3 (olaratumab) and other PDGFRα antagonists are useful for treating tumours characterized by autocrine and/or paracrine stimulation of PDGFRα.

The methods of the present invention also find utility in monitoring the response of a subject to a treatment for cancer. The present invention includes monitoring the efficacy of a treatment for a PDGFRα-expressing cancer, wherein the treatment includes but is not limited to administration of any one or more of: surgery, chemotherapy, immunotherapy, autologous stem cell transfer (ASCT), external beam radiation, immuno-radiation an inhibitor of PDGFRalpha or combinations thereof.

Typically the methods for monitoring response to treatment include a comparison of an amount of detectable cancer after or during treatment compared to the amount of detectable cancer prior to treatment or at an earlier time point during treatment. In accordance with the methods of the present invention, this typically involves comparing the amount of radiolabelled olaratumab detected in the patient or cancer at different time points.

In certain embodiments, the detecting or determining or imaging methods described herein may result in data which is used to compile training data for development of a deep learning algorithm for enabling artificial-intelligence based self-learning for diagnosis and/or staging of the cancer. This includes the use of such artificial-intelligence based methods for compiling data from the individual patient over the course of their therapy in order to assess progress of treatment.

Further still, the methods may involve using deep learning algorithms to stratify or classify a cancer, or a subject having cancer, as likely or unlikely to respond to treatment with an inhibitor of PDGFRalpha. In certain examples, the training data (or reference data set upon which the deep learning algorithm is based), may comprise data from one or more individuals who previously responded to treatment with an inhibitor of PDGFRalpha; in further examples, the training data (or reference data set upon which the deep learning algorithm is based), may comprise data from one or more individuals who did not respond to treatment with an inhibitor of PDGFRalpha. The training data may comprise data from a combination of patients who had different responses to treatment with an inhibitor of PDGFRalpha.

In certain embodiments, the imaging or detecting data obtained from the methods described herein may be assessed relative to a disease classification model that has been generated using machine learning techniques using training data (or reference data set upon which the deep learning algorithm is based), comprising data from one or more individuals who previously responded to treatment with an inhibitor of PDGFRalpha; and/or from one or more individuals who did not respond to treatment with an inhibitor of PDGFRalpha.

Accordingly, AI-based approaches described herein can serve as a platform on which a variety of tools and techniques for detecting, evaluating, and predicting cancer status for patients can be built. Moreover, since the AI-based tools as described herein can identify a variety of important tissue regions across an entire body of a patient, and then transfer those identifications (e.g., segmentation maps) from anatomical images to various functional images, they can be used as a building block for analysis techniques based on a variety of different imaging methods that obtain contrast via a variety of different radiopharmaceuticals.

The use of AI-based technologies using machine learning techniques (such as Convolution Neural Networds) and the like to stratify patients to treatment, monitor disease progression, or assess or diagnose a cancer based on the imaging techniques described herein, are generally known in the art, and are described for example in WO2020144134 and U.S. Pat. No. 11,443,201, incorporated herein by reference.

As used herein, a level or amount that is higher than the reference dataset or amount generally refers to an amount that is at least more than about 10%, at least about 20%, at least about 30%, at least about 40%, at least about 50%, at least about 60%, at least about 70%, at least about 80%, at least about 90% or at least 100% or greater.

As used herein, a level or amount that is lower than the reference dataset or amount generally refers to an amount that is at least about 10%, at least about 20%, at least about 30%, at least about 40%, at least about 50%, at least about 60%, at least about 70%, at least about 80%, at least about 90% or at least 100% or more, lower than the amount in the reference dataset.

The same, higher or lower amount of level or amount generally refers to an amount that differs by no more than about 5% preferably no more than 10% from the reference dataset.

The skilled person will be familiar with methods for determining statically significant differences between the level or amount of detectable radiolabelled olaratumab for the purposes of making the comparison required for the present invention.

In accordance with the present specification, determining the response to treatment of a PDGFRalpha-expressing cancer or a subject having a PDGFRalpha-expressing cancer (or where the individual is considered to have responded to treatment), includes stabilisation of the disease or the slowing down or cessation of disease progression. ‘Response to treatment’ refers to therapeutic treatment wherein the object is to slow down (lessen) an undesired physiological change or disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. Treatment can also mean prolonging survival as compared to expected survival if not receiving treatment. Treatment may not necessarily result in the complete clearance of a disease or disorder but may reduce or minimise complications and side effects of infection and the progression of a disease or disorder.

As used herein, a positive response of an individual to a treatment includes an increase in the progression free survival of the individual. Alternatively, a positive response of an individual to a treatment includes an increase in the overall survival of the individual. Accordingly, the present invention also finds utility in predicting the overall survival or progression free survival of an individual receiving/requiring treatment for a PDGFRα-expressing cancer.

As used herein “overall survival” (OS) refers to the length of time from either the date of diagnosis or the start of treatment for a cancer, that patients diagnosed with the cancer are still alive. In a clinical trial, measuring the overall survival is one way to see how well a new treatment works.

‘Overall survival’ or ‘OS’ is well known to one of skill in the art and refers to the fate of the patient after an event, preferably after the start or end of a treatment regime, despite the possibility that the cause of death in a patient is not directly due to the effects of the disease (cancer). In other words, it refers to the prognosis that the patient will not die because of PDGFRα-expressing cancer, preferably within at least 1 year, at least 2 years, at least 3 years, at least 4 years, at least 5 years, at least 10 years or at least 15 years.

As used herein “progression free survival” (PFS) refers to the length of time during and after the treatment of a disease, such as cancer, that a patient lives with the disease but it does not get worse. In a clinical trial, measuring the progression-free survival is one way to see how well a new treatment works.

‘Responding to a treatment regime’ refers to a clinically or biochemically favourable detectable response to a treatment. Typically, a favourable response is survival measured at a later time point after treatment, for example, 1, 2, 3, or 4 years post treatment.

### Kits

In another embodiment there is provided a kit or article of manufacture including a radiolabelled olaratumab or antigen binding fragment, as described above, preferably provided for use in a method or use outlined herein.

Optionally the kit further comprises a label or package insert with instructions for use.

The kit or “article of manufacture” may comprise a container and a label or package insert on or associated with the container. Suitable containers include, for example, bottles, vials, syringes, blister pack, etc. The containers may be formed from a variety of materials such as glass or plastic. The container holds a diagnostic composition which is effective for imaging or detecting as described herein and may have a sterile access port (for example the container may be an intravenous solution bag or a vial having a stopper pierceable by a hypodermic injection needle). The label or package insert indicates that the composition is used for diagnosing or detecting the condition of choice. In one embodiment, the label or package insert includes instructions for use.

Alternatively, or additionally, the kit may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In certain embodiments the diagnostic composition may be provided in the form of a device, disposable or reusable, including a receptacle for holding the diagnostic composition. In one embodiment, the device is a syringe. The device may hold 1-2 mL of the therapeutic composition. The diagnostic composition may be provided in the device in a state that is ready for use or in a state requiring mixing or addition of further components.

In other embodiments there is provided a kit for use in a diagnostic application mentioned above, the kit including:
a container holding a diagnostic composition in the form of a radiolabelled olaratumab or antigen binding fragment thereof;
a label or package insert with instructions for use.

The kit may comprise (a) a diagnostic composition; and (b) a second container with a second diagnostic agent or second label contained therein. It may further include other materials desirable from a commercial and user standpoint, including other buffers, diluents, filters etc.

It will be understood that the invention disclosed and defined in this specification extends to all alternative combinations of two or more of the individual features mentioned or evident from the text or drawings. All of these different combinations constitute various alternative aspects of the invention.

The examples that follow are intended to illustrate but in no way limit the present invention.

### EXAMPLES

### Example 1: Synthesis and Characterisation of Bioconjugates

An olaratumab immunoglobulin antibody comprising a VH as set forth in SEQ ID NO: 4 and a VL as set forth in SEQ ID 12 was used to generate olaratumab-bioconjugates in the form of DOTA-Olaratumab and DFOsq-Olaratumab.

DOTA-Olaratumab and DFOsq-Olaratumab bioconjugation was performed using standard methods. 30 mg of each conjugate was synthesized, and analytical characterisation of the conjugates was undertaken.

TABLE 2
Conjugation reaction of Olaratumab with DOTA-NHS ester

| Reagents | Antibody: 0.2 mg | |
|---|---|---|
| | DOTA-NHS ester: MW 501.5 g/mol | |
| | @35 mg/mL; 25-30 eq per mAb | |
| | Buffer: 100 mM sodium borate, pH 9 | |
| Reaction conditions | Time | 2 hrs |
| | Temperature | 22° C. |

DOTA-NHS conjugate demonstrated high monomeric purity (>98%) and values of 4.4 and 4.3 for DOTA-Olaratumab 1 and DOTA-Olaratumab 2 (generated using two different cell culture methods, respectively); value of degree of antibody labelling (DOL) (data not shown).

TABLE 3
Conditions of the conjugation reaction of Olaratumab with DFO-sq

| Reagents | Antibody: 0.2 mg | |
|---|---|---|
| | DFO-sq: MW 684.8 g/mol @5 mg/mL; | |
| | 16-18 eq per mAb | |
| | Buffer: 100 mM sodium borate, pH 9 | |
| | Organic co-solvent: 10% DMSO | |
| Reaction conditions | Time | 3 hrs |
| | Temperature | 22° C. |

Size exclusion chromatography (SEC) screening of DFOsq-olaratumab conjugate with DFPsq indicated high monomeric purity (>98%) after conjugation (data not shown).

DFOsq-conjugated olaratumab was further assessed for the characteristics listed below in table 3.

TABLE 4
analytical characterisation of DOTA and DFOsq bioconjugates
produced in stable and transient transfection cell lines

| | Assay | DOTA | DFOsq |
|---|---|---|---|
| Batch Number | | TEPH-2187- | TEPH-2187- |
| | | MI002-2 | MI002-3 |
| Purity | SEC | >99.5% monomeric | >99.1% monomeric |
| | Endotoxin | 0.12 EU/mg | 0.03 EU/mg |
| Identity and | LC-MS | DAR 3.8 | DAR 2-3 |
| DOL | | | |
| Concentration | UV | 2.5 mg/mL | 3.44 mg/mL |
| Formulation | | 50 mM Sodium | PBS, |
| and storage | | acetate, 150 mM | pH 7.1 −80° C. |
| | | sodium chloride, | |
| | | pH 5.5 −80° C. | |

### Biacore Antibody and Conjugate Screening for Binding to PDGFRα

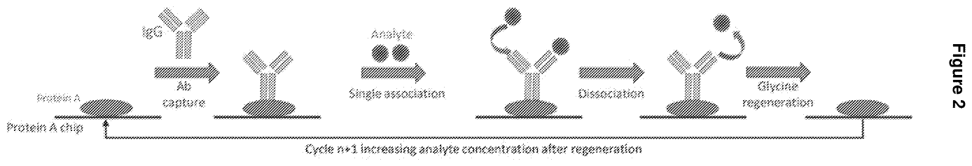

Comparative binding of the produced conjugates and Olaratumab to PDGFRα (Sino Biological, #10556-H08H, Lot #LC15MY0708) was tested using Biacore MCK, surface plasmon resonance-based technology (FIG. 2). Instrument used was Biacore T200. Running buffer: HBS-P+ buffer containing 1 mg/ml BSA. Chip: Protein A capture Sensor chip. Analysis temperature: 25° C. Flow rate: 30 μl/min. Ligand: purified antibody ~80 RU at 10 μl/min. Analyte: human PDGFRα. Dilution range: 8 point 3-fold dilution from 270 nM to 0.123 nM. Analyte injection time: 240 s. Analyte dissociation time: 1200 s. Regeneration: glycine pH 1.5. Analysis: 1:1 binding with double reference subtraction.

The results indicated that all tested antibodies bind with similar affinity to PDGFRα (Table 5).

TABLE 5
Characterisation of naked olaratumab, DOTA- and
DFOsq-conjugated olaratumab binding to PDGFRα

| | Kₐ | K_{d} | K_{D} | | Chi² |
|---|---|---|---|---|---|
| Ligand | (1/Ms) | (1/s) | (M) | R_{MAX} | (RU²) |
| Olaratumab | 3.39E+05 | 2.07E−03 | 6.11E−09 | 37.6 | 0.259 |
| DOTA- | 3.88E+05 | 2.09E−03 | 5.38E−09 | 32.3 | 0.305 |
| Olaratumab | | | | | |
| DFOSq- | 3.75E+05 | 2.10E−03 | 5.61E−09 | 35.8 | 0.328 |
| Olaratumab | | | | | |
| Olaratumab | 3.31E+05 | 1.93E−03 | 5.83E−09 | 43.8 | 0.362 |
| DOTA- | 3.82E+05 | 2.07E−03 | 5.41E−09 | 35.9 | 0.181 |
| Olaratumab | | | | | |
| DFOsq- | 3.62E+05 | 2.01E−03 | 5.56E−09 | 32.1 | 0.203 |
| Olaratumab | | | | | |

### Example 2: Validation of PDGFRα Expression in Selected Cell Lines

Selection of the cell lines for the subsequent tests in xenograft tumour models was based on data published by Lowery et al, 2017, where efficacy of olaratumab has been demonstrated in HuO9 (osteosarcoma) and A-204 (embryonal rhabdomyosarcoma) cell lines.

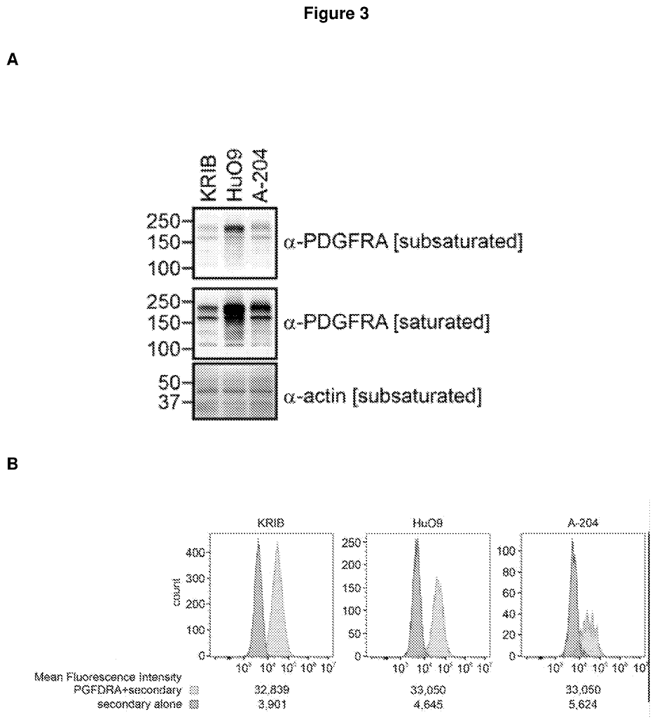

FIG. 3 shows total and surface PDGFRA expression was ascertained with in KRIB, HuO9 and A-204 sarcoma cell lines. All three cell lines expressed PDGFRA, which was most abundant in the HuO9; however, HuO9 cells were found to have smaller proportion of the total amount of PDGFRAA localised to the plasma membrane.

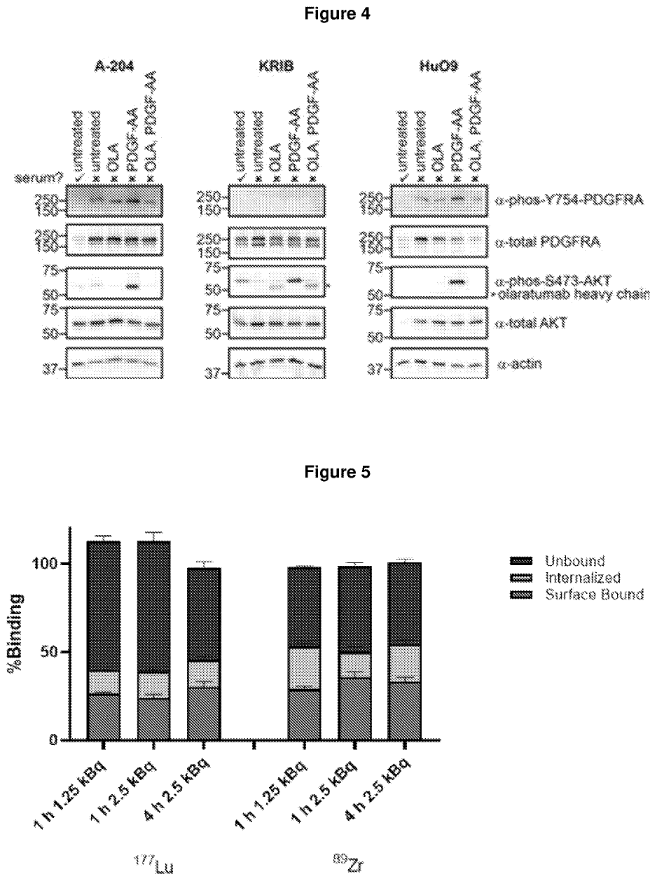

PDGF-AA stimulation of all three cell lines triggered phosphorylation of AKT on serine 473. This was abolished by prior incubation with olaratumab (FIG. 4).

### Example 3: Development of In Vivo Xenograft Model

KRIB and A204 cell line growth properties were tested in a xenograft model in Balb/c Nude mice. Both cell lines showed reproducible growth and good “take-rates”. The A204 cell clone was selected for subsequent biodistribution studies in view of greater radiosensitivity of that cell line to ionising radiation.

Radiolabelling of DFOSq-Olaratumab yielded 94% radiochemical purity as assessed with Quality Control methods. Surface binding and internalisation of the radiolabelled antibodies to the chosen reference cell line was assessed in binding and Immunoreactive Fraction (IRF) assay.

Ratio of activity to cells was based on initial pilot which showed similar saturated binding with 1.25 and 2.5 kBq/5 million cells at 1 hour, and no binding to negative control cells (specific activity at 1 MBq/4 μg). Saturated binding (IRF) was established to be at around 40% in the A204 cells at 1 h and ~3% binding in the negative control line (HCC827) (FIG. 5, A). 30-50% of the total binding was found to be internalised after 1 h (FIG. 5, B).

### Example 4: Imaging and Biodistribution Studies

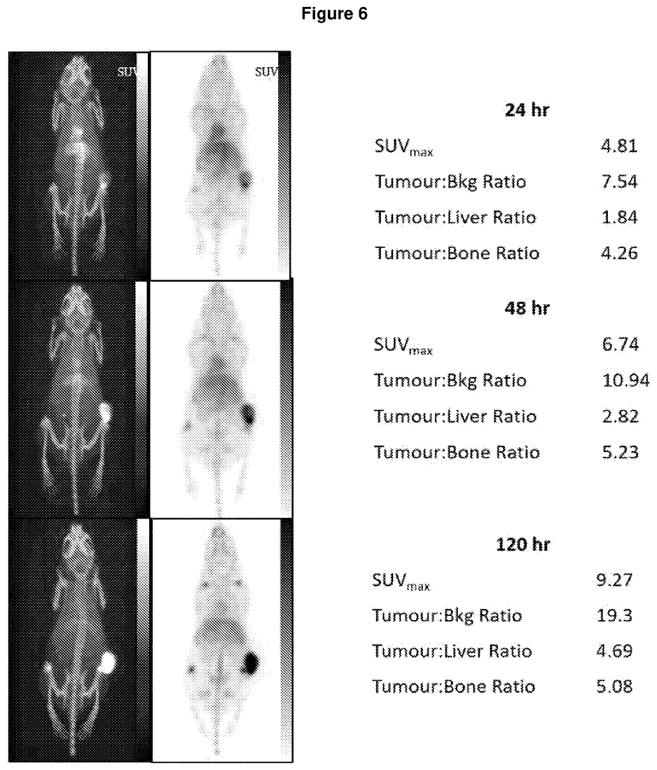

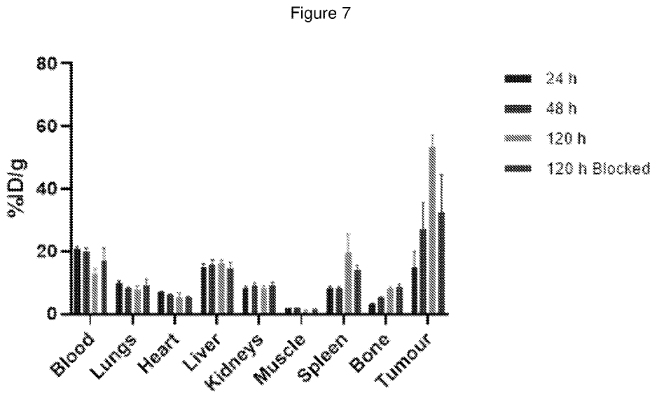

Balb/c nude mice (N=4) were injected with 5.03 MBq at antibody mass dose of 20 μg and PET images were taken at 1, 2, 4 and 6 days after injection (FIG. 6). For the blocking 344 μg (a nominal 17-fold excess) of unlabelled antibody conjugate was co-injected. Blocking mice were imaged at 1-day post-injection and re-imaged at the later timepoint(s) or harvested by biodistribution. Mice were euthanised and tissues harvested for biodistribution at 1, 2 and 4 days post injection (n=4 per time point) (FIG. 7).

Imaging and biodistribution analyses demonstrated that Olaratumab is targeted to tumour with high tumour/background ratio over the tested period. High tumour uptake of ~55% of ID/g was observed at 120 hrs post-injection and demonstrated tumour/background ratio ~20.

### Example 5: Clinical Trial of ⁸⁹Zr-Olaratumab (⁸⁹Zr-TLX300-CDx) in Patients with Soft Tissue Sarcoma

### Objectives

### Primary Objectives:

evaluate clinical safety and tolerability of ⁸⁹Zr-TLX300-CDx
evaluate pharmacokinetics biodistribution and radiation dosimetry of ⁸⁹Zr-TLX300-CDx

### Secondary Objectives

determine a suitable antibody mass for administration

### Exploratory

evaluate the correlation between tumour uptake of ⁸⁹Zr-TLX300-CDx and PDGFRα expression
compare the tumour uptake and detection of lesions between ⁸⁹Zr-TLX300-CDx and standard of care imaging

### Endpoints

### Primary Endpoints

adverse events (AEs), change in clinical laboratory tests, ECG, physical examination, and vital signals
⁸⁹Zr-TLX300-CDx uptake within tumour and organs
blood radioactivity
blood olaratumab levels

### Secondary Endpoints

uptake of radioactivity in tumour lesions and organs
whole body absorbed and effective radiation dose
blood olaratumab levels

### Exploratory Endpoints

expression of PDGFRα in prior/archival tumour tissue and/or surgically resected tumour samples
⁸⁹Zr-TLX300-CDx uptake in tumour lesions
⁸⁹Zr-TLX300-CDx uptake within tumours and organs
localisation of tumour deposits on conventional imaging (CT, MRI and/or FDG-PET)

### Methodology:

### Study Design

Open-label, first-in-human Phase 1 imaging study.

The target population is histologically confirmed STS. The study includes three parts: proof-of-concept tumour targeting of ⁸⁹Zr-TLX300-CDx (Part A), assessment of ⁸⁹Zr-TLX300-CDx biodistribution and tumour uptake (Part B) and radiation dosimetry (Part C).

All participants undergo a formal screening visit (Visit 1), during which the study schedule is planned, and participant consent obtained. The Screening Period of up to 30 days (Day −30 to Day −1) includes study qualification assessments.

### Part A—Proof-of-Concept Tumour Targeting of ⁸⁹Zr-TLX300-CDx

Three (3) participants with PDGFRα-positive STS are recruited to Cohort 1 to receive a single administration of 37 MBq (±10%)⁸⁹Zr-TLX300-CDx, containing a mass dose of 25 mg of olaratumab.

The study team evaluate the emerging Positron Emission Tomography (PET)/Computerised Tomography (CT) data from Cohort 1 and determine if enrolment of additional participants is needed to refine the administered mass dose level. Additional participants may be recruited to Cohort 1 (for a total of 6 participants in Cohort 1), or to additional cohorts of 3-6 participants, where the cohorts may cover different antibody mass doses ranging from 1 to 100 mg of olaratumab, each with an activity of 37 MBq (±10%) ⁸⁹-zirconium.

Screening window: up to 30 days before administration of ⁸⁹Zr-TLX300-CDx

### Study Period:

intervention: ⁸⁹Zr-TLX300-CDx administered on Day 1 as a slow, 3 to 5 min intravenous (IV) bolus injection.
imaging period: static whole-body PET/CT imaging at 5 days±1 day post-injection, with optional additional timepoint at 4 h±0.5 h post-injection.
blood radioactivity and total antibody quantitation: pre-injection, 4 h±0.5 h, 5 days±1 day post-injection
EOS visit within 30 days of Visit 2 (i.e., ⁸⁹Zr-TLX300-CDx administration).

Part A concludes once a suitable antibody mass dose has been identified by the study team upon review of the emerging PET/CT data. A maximum of 15 participants are enrolled in Part A.

Safety data is reviewed by an independent SRC after Part A is completed. A participant is deemed evaluable for safety if they have been administered ⁸⁹Zr-TLX300-CDx and completed the EOS visit.

Once Part A is completed and antibody mass dose selected, Parts B and C open in parallel for enrolment.

### Part B—Assessment of ⁸⁹Zr-TLX300-CDx Biodistribution and Tumour Uptake

Up to 20 participants are enrolled in Part B (regardless of their PDGFRα expression status on prior/archival tissue) to assess biodistribution and potential variations in tumour uptake.

Once the first 10 participants are enrolled in Part B, rates of tumour uptake are calculated.

Screening window: up to 30 days before administration of ⁸⁹Zr-TLX300-CDx

### Study Period:

intervention: ⁸⁹Zr-TLX300-CDx administered on Day 1 as a slow, 3 to 5 min intravenous (IV) bolus injection.
imaging period: static whole-body PET/CT imaging at 5 days±1 day post-injection, with optional additional timepoint at 4 h±0.5 h post-injection.
blood radioactivity and total antibody quantitation: pre-injection, 4 h±0.5 h and 5 days±1 day post-injection.
EOS visit within 30 days of Visit 2 (i.e., ⁸⁹Zr-TLX300-CDx administration).

### Part C—Radiation Dosimetry

Six (6) participants (3 males, 3 females) are initially be enrolled in Part C at selected sites for radiation dosimetry. Part C participants are enrolled regardless of their PDGFRα expression status on prior/archival tissue. The study team evaluates the emerging PET/CT data and determines if enrolment of additional participants is needed (a maximum of 15 participants may be enrolled in Part C).

Screening window: up to 30 days before administration of 89Zr-TLX300-CDx

### Study Period:

intervention: ⁸⁹Zr-TLX300-CDx administered on Day 1 as a slow, 3 to 5 min IV bolus injection.
imaging period: sequential static whole-body PET/CT imaging at 24 h±4 h, 3 days±1 day and 6 days±1 day post-injection, with optional dynamic imaging 15 min±2 min post-injection at selected sites and optional static imaging at 4 h±0.5 h post-injection at any sites
blood radioactivity and total antibody quantitation: pre-injection, 4 h±0.5 h, 24 h±4 h, 3 days±1 day and 6 days±1 day post-injection, with optional additional timepoint at 7 h±1 h post-injection.
EOS visit within 30 days of Visit 2 (i.e., ⁸⁹Zr-TLX300-CDx administration)

Moreover, for participants where the target tumour is resected within 30 days of Visit 2 (i.e., ⁸⁹Zr-TLX300-CDx administration), resected tumour samples will be collected for exploratory analyses.

### End of Study (EOS) Visit Will Take Place:

after the last imaging timepoint (can be on the same day)
within 30 days of Visit 2 (i.e., 89Zr-TLX300-CDx administration)
before the surgical resection of the target tumour (if applicable, and can be on the same day as the surgery).

### Inclusion Criteria

Participants are eligible to be included in the study only if all of the following criteria apply:
1. ≥18 years of age at the time of signing the informed consent.
2. Histologically confirmed diagnosis of soft tissue sarcoma (STS)
3. At least one mass of >2 cm in largest diameter seen on standard of care imaging (CT, MRI and/or FDG-PET).
4. For Part A: Participants must have tumour PDGFRα expression confirmed by IHC. Participants must have consented to provide prior/archival FFPE tumour tissue. For Parts B and C: all participants will be included regardless of their PDGFRα expression status on prior/archival tissue (unless otherwise specified). Participants must have consented to provide prior/archival FFPE tumour tissue.
5. Adequate haematologic function as defined by an absolute neutrophil count (ANC) ≥1500/μL, haemoglobin ≥9.0 g/dL, and a platelet count of 100,000/μL obtained.
6. Adequate hepatic function as defined by a total bilirubin ≤1.5 mg/dL, and aspartate transaminase (AST) and alanine transaminase (ALT) ≤3.0 times the upper limit of normal (ULN).
7. Adequate renal function as defined by serum creatinine 1.5× the institutional ULN. If creatinine is above the ULN, the participant's creatinine clearance is 45 mL/min.
8. Eastern Cooperative Oncology Group (ECOG) performance status of 0 to 2.
9. Life expectancy of at least 6 months.
10. Female participants of childbearing potential must have negative pregnancy tests at screening, as well as confirmation of negative pregnancy test result within 24 hours prior to receiving 89Zr-TLX300-CDx. Female participants of childbearing potential or male participants with female partners of childbearing potential must:
   a. be willing to practice full and true sexual abstinence; or
   b. be surgically/permanently sterile or with a history of hysterectomy for women; or
   c. be willing to practice highly effective contraception by using: a non-oral, injected or implanted non-oestrogen progesterone based hormonal method, male condom, vaginal diaphragm, cervical cap, intrauterine device, for 3 months after the administration of 89Zr-TLX300-CDx.
11. Capable of giving signed informed consent which includes compliance with the requirements and restrictions listed in the informed consent form (ICF) and in this protocol.

### Exclusion Criteria

Participants are excluded from the study if any of the following criteria apply:
1. Known or suspected hypersensitivity to olaratumab, DFOsq, 89Zr or any of the excipients.
2. IgE antibodies against galactose-α-1,3-galactose (α-Gal) above the upper limit of normal, >0.7 kU/L.
3. Exposure to any experimental diagnostic or therapeutic drug within 30 days from the date of planned administration of 89Zr-TLX300-CDx.
4. Surgery ≤2 weeks prior to the administration of 89Zr-TLX300-CDx or significant ongoing complications of surgery. Biopsy ≤2 weeks prior to the administration of 89Zr-TLX300-CDx is allowed.
5. Exposure to any radiopharmaceutical within 10 half-lives prior to the administration of 89Zr-TLX300-CDx.
6. Ongoing toxicity Grade 2 or higher from previous standard or investigational therapies (Common Terminology Criteria for Adverse Events [CTCAE] version 5).
7. Planned to commence systemic antineoplastic therapies, immunotherapy, targeted therapy, radiotherapy and/or surgery for the period between administration of ⁸⁹Zr-TLX300-CDx and last imaging timepoint.
8. Serious non-malignant disease (e.g., psychiatric, infectious, autoimmune or metabolic) or any disease that may interfere with the objectives of the study or with the safety or compliance of the participant, as judged by the Investigator.
9. Pregnant or lactating women.
10. Participants unable to declare meaningful informed consent on their own (e.g., with legal guardian for mental disorders) or unable to tolerate the study procedures.

### Example 6: Imaging of Ovarian Cancer

Imaging studies using ⁸⁹-Zr-olaratumab is conducted to assess the ability of the imaging reagent to detect ovarian cancers.

Firstly, the experiments will test the ability of ⁸⁹-Zr-olaratumab to bind to various ovarian cancer cell lines. These results will demonstrate the ability of olaratumab to bind to various ovarian cancer cell types which express PDGFRα, although the degree of in vitro binding is variable.

Subsequently, mice bearing A2780 cell line xenograft (approximately n=4) will be intravenously administered with ⁸⁹-Zr-olaratumab.

At 24 hours and 72 hours following intravenous injection of ⁸⁹-Zr-olaratumab into the xenograft-bearing mice, the mice are subjected to PET/CT scanning to determine the ability of the ⁸⁹Zr-olaratumab antibodies to detect the ovarian cancer cells in vivo.

The results will show that ⁸⁹Zr-olaratumab antibodies is able to bind to the ovarian tumour xenografts. In other words, the results will demonstrate that ⁸⁹Zr-olaratumab antibodies are able to bind to target ovarian tumour cells in an in vivo context and with similar affinity to in vitro.

These results will indicate that ⁸⁹Zr-olaratumab antibodies are suitable for use in producing an image of ovarian cancer types in vivo, and is therefore useful as a non-invasive diagnostic reagent for diagnosis and detection of cancers other than soft tissue sarcomas.

1. An olaratumab antibody or olaratumab antibody bioconjugate, suitable for radiolabelling with a diagnostic radioisotope.

2. The olaratumab bioconjugate of claim 1, wherein the bioconjugate comprises an olaratumab antibody conjugated to a chelating or linker group selected from: TMT (6,6″-bis[N,N″,N′″-tetra(carboxymethyl)aminomethyl)-4′-(3-amino-4-methoxyphenyl)-2,2′:6′,2″-terpyridine), DOTA (1,4,7,10-tetraazacyclododecane-NN′,N″(N′″-tetraacetic acid), TCMC, DO3A, CB-DO2A, NOTA, Diamsar, DTPA, CHX-A″-DTPA, olaratumab-TETE, Te2A, HBED, DFO, DFOsq, DFO-NCS and HOPO.

3. An olaratumab antibody or antigen binding fragment thereof, conjugated to a radioisotope suitable for in vivo imaging, for:
the detection or imaging of PDGFRalpha-expressing tumours in a subject;
for the diagnosis of a PDGFRalpha-expressing cancer in a subject;
for producing an image of a PDGFRalpha-expressing cancer in a subject;
for classifying a cancer as sensitive to treatment with a PDGFRalpha-inhibitor;
for classifying or selecting a patient for eligibility for cancer therapy with an inhibitor of PDGFRalpha; or
for monitoring the response to treatment of a PDGFRalpha-expressing cancer or a subject having a PDGFRalpha-expressing cancer.

4. The olaratumab antibody or antigen binding fragment thereof, or antibody bioconjugate of any one of claims 1 to 3, comprising an antigen binding domain that competitively inhibits the binding of an antibody comprising a VH comprising a sequence as set forth in SEQ ID NO: 4 and a VL comprising a sequence as set forth in SEQ ID NO: 12.

5. The olaratumab antibody or antigen binding fragment thereof or bioconjugate of any one of claims 1 to 4, wherein the antibody or fragment thereof comprises an HCDR1, a HCDR2 and an HCDR3 of an antigen binding domain having a variable heavy chain as defined in in SEQ ID NO: 4 and a LCDR1, LCDR2 and LCDR3 of an antigen binding domain having a VL as defined in SEQ ID NO: 12.

6. The olaratumab antibody or antigen binding fragment thereof of any one of claims 1 to 5, wherein the antibody or antigen binding fragment comprises an antigen binding domain comprising:
(i) a VH comprising a complementarity determining region (CDR) 1 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical to a sequence set forth in SEQ ID NO: 1, a CDR2 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical to a sequence set in SEQ ID NO: 2, and a CDR3 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical to a sequence set forth in SEQ ID NO: 3;
(ii) a VH comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical to a sequence set forth in SEQ ID NO: 4;
(iii) a VL comprising a CDR1 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical to a sequence set forth in SEQ ID NO: 9, a CDR2 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical to a sequence set forth in SEQ ID NO: 10 and a CDR3 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical to a sequence set forth in SEQ ID NO: 11;
(iv) a VL comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical to a sequence set forth in SEQ ID NO: 12;
(v) a VH comprising a CDR1 comprising a sequence set forth in SEQ ID NO: 1, a CDR2 comprising a sequence set forth in SEQ ID NO: 2 and a CDR3 comprising a sequence set forth in SEQ ID NO: 3;
(vi) a VH comprising a sequence set forth in SEQ ID NO: 4;
(vii) a VL comprising a CDR1 comprising a sequence set forth in SEQ ID NO: 9, a CDR2 comprising a sequence set forth in SEQ ID NO: 10 and a CDR3 comprising a sequence set forth in SEQ ID NO: 11;
(viii) a VL comprising a sequence set forth in SEQ ID NO: 12;
(ix) a VH comprising a CDR1 comprising a sequence set forth in SEQ ID NO: 1, a CDR2 comprising a sequence set forth between in SEQ ID NO: 2, and a CDR3 comprising a sequence set forth in SEQ ID NO: 3; and a VL comprising a CDR1 comprising a sequence set SEQ ID NO: 9, a CDR2 comprising a sequence set forth in SEQ ID NO: 10, and a CDR3 comprising a sequence set forth in SEQ ID NO: 11; or
(x) a VH comprising a sequence set forth in SEQ ID NO: 4, and a VL comprising a sequence set forth in SEQ ID NO: 12.

7. The olaratumab antibody or antigen binding fragment thereof of claim 6, further comprising:
(i) a VH comprising a framework region (FR) 1 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in SEQ ID NO: 5; a FR2 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in SEQ ID NO: 6; a FR3 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in SEQ ID NO: 7; a FR4 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in SEQ ID NO: 8; and
(ii) a VL comprising a framework region (FR) 1 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in SEQ ID NO: 13; a FR2 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in SEQ ID NO: 14; a FR3 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in SEQ ID NO: 15; a FR4 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in SEQ ID NO: 16.

8. The olaratumab antibody or antigen binding fragment thereof of claim 6, further comprising:
(i) a VH comprising a framework region (FR) 1 comprising or consisting of a sequence as set forth in SEQ ID NO: 5; a FR2 comprising or consisting of a sequence as set forth in SEQ ID NO: 6; a FR3 comprising or consisting of a sequence as set forth in SEQ ID NO: 7; a FR4 comprising or consisting of a sequence as set forth in SEQ ID NO: 8, and
(ii) a VL comprising a framework region (FR) 1 comprising or consisting of a sequence as set forth in SEQ ID NO: 13; a FR2 comprising or consisting of a sequence as set forth in SEQ ID NO: 14; a FR3 comprising or consisting of a sequence as set forth in SEQ ID NO: 15; a FR4 comprising or consisting of a sequence as set forth in SEQ ID NO: 16.

9. The olaratumab antibody or antigen binding fragment thereof of any one of claims 1 to 8, wherein the antibody or antigen binding fragment thereof comprises a VH comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical to a sequence set forth in SEQ ID NO: 4; and/or a VL comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical to a sequence set forth in SEQ ID NO: 12; wherein the sequence variation between the VH and VL and the sequence of SEQ ID NO: 4 and 12 is not in the CDRs and wherein the antibody or antigen binding fragment thereof retains the ability to bind to PDGFRalpha.

10. The olaratumab antibody or antigen binding fragment thereof of any one of claims 1 to 9, wherein the antibody or antigen binding fragment thereof comprises a VH and/or a VL comprising no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19 or no more than 20 amino acid residue substitutions, deletions or additions, compared to the amino acid sequences as set forth in SEQ ID NO: 4 or 12, respectively; wherein the amino acid substitutions, deletions or additions are not in the CDRs and the antibody or antigen binding fragment thereof retains the ability to bind to PDGFRalpha.

11. The olaratumab antibody or antigen binding fragment thereof of any one of claims 1 to 10, wherein the antibody or antigen binding fragment is in the form of:
(i) a single domain antibody (sdAb);
(ii) a single chain Fv fragment (scFv);
(iii) a dimeric scFv (di-scFv); or
(iv) one of (ii) or (iii) linked to a constant region of an antibody, Fc or a heavy chain constant domain (CH) 2 and/or CH3.

12. The olaratumab antibody or antigen binding fragment thereof of any one of claims 1 to 10, wherein the antibody or antigen binding fragment is in the form of:
(i) a diabody;
(ii) a triabody;
(iii) a tetrabody;
(iv) a Fab;
(v) a F(ab′)2;
(vi) a Fv;
(vii) a bispecific antibody or other form of multispecific antibody; or
(viii) one of (i) to (vii) linked to a constant region of an antibody, Fc or a heavy chain constant domain (CH) 2 and/or CH3.

13. The olaratumab antibody or antigen binding fragment thereof of any one of claims 1 to 12, wherein the antibody or antigen binding fragment is in the form of an immunoglobulin such as an IgG1, IgG2, IgG3 or IgG4.

14. The olaratumab antibody or antigen binding fragment thereof of any one of claims 1 to 13, wherein the olaratumab antibody comprises a heavy chain constant region as defined in SEQ ID NO: 17 and/or a light chain constant region as defined in SEQ ID NO: 18.

15. The olaratumab antibody or antigen binding fragment thereof of any one of claims 1 to 14, wherein the olaratumab antibody comprises a heavy chain as set forth in SEQ ID NO: 19 and/or a light chain as set forth in SEQ ID NO: 20.

16. The olaratumab antibody or antigen binding fragment thereof of any one of claims 13 to 15, wherein the antibody or antigen binding fragment thereof comprises one or more amino acid substitutions in the constant region, so as to reduce the in vivo half-life of the antibody.

17. The olaratumab antibody or antigen binding fragment thereof of any one of claims 13 to 16, wherein the antibody or antigen binding fragment thereof comprises substitutions at one or more of residues His310, His435, Tyr436 and Ile253 of the constant region of the antibody (Kabat EU index numbering).

18. The olaratumab antibody or antigen binding fragment thereof of any one of claims 13 to 17 wherein the antibody or antigen binding fragment thereof comprises an amino acid substitution at position 310 selected from alanine, glutamic acid or glutamine (Kabat EU index numbering).

19. The olaratumab antibody or antigen binding fragment thereof of any one of claims 13 to 18, wherein the antibody or antigen binding fragment thereof comprises an amino acid substitution at position 435 selected from arginine, glutamine or alanine (Kabat EU index numbering).

20. The olaratumab antibody or antigen binding fragment thereof of any one of claims 13 to 19, wherein the antibody or antigen binding fragment thereof comprises an alanine residue at position 310 and glutamine residue at position 435 (Kabat EU index numbering).

21. The olaratumab antibody or antigen binding fragment thereof of any one of claims 13 to 19, wherein the antibody or antigen binding protein thereof comprises an amino acid substitution at position Ser228 and/or Leu235, preferably wherein the substitution is Leu235Glu and/or Ser228Pro.

22. The olaratumab antibody or antigen binding fragment thereof of any one of claims 16 to 21, wherein the binding affinity for FcRn and/or the serum half-life of the antibody or antigen binding fragment thereof is decreased by at least about 2-fold, 3-fold, 4-fold, 5-fold, 10-fold, 15-fold, 20-fold, 25-fold, 30-fold, 40-fold, 50-fold, 60-fold, 70-fold, 80-fold, 90-fold, or 100-fold; or wherein the binding affinity for FcRn and/or the serum half-life of said modified antibody is reduced by at least about 20%, 30%, 40%, 50%, 60%, 70%, 80%, 85%, 90%, 95%, 97%, 98%, or 99%, compared to an immunoglobulin that does not comprise the same substitutions.

23. The olaratumab antibody or antigen binding fragment thereof of any one of claims 3 to 12, wherein the antibody or antigen binding fragment thereof comprises a radioisotope suitable for in vivo imaging methods for detection of tumour or cancer masses.

24. The olaratumab antibody or antigen binding fragment thereof of claim 23, wherein the radioisotope is selected from: fluorine-18 (¹⁸F), gallium-67 and gallium-68 (⁶⁷Ga and ⁶⁸Ga), indium-111 (¹¹¹In), iodine-123 and iodine-124, technetium-99 (^{99m}Tc), terbium-155 and terbium-159 (¹⁵⁵Tb and ¹⁵⁹Tb) and zirconium-89 (⁸⁹Zr).

25. The olaratumab antibody or antigen binding fragment thereof of any one of claims 23 to 24, wherein the radioisotope is conjugated to the antibody or antigen binding fragment thereof directly e.g. by halogenation of amino acid residues.

26. The olaratumab or antigen binding fragment thereof of any one of claims 23 to 24, wherein the radioisotope is linked indirectly to the antibody or antigen binding fragment thereof, for example via a chelator or other linking moiety.

27. The olaratumab of claim 26, wherein the antibody or antigen binding fragment thereof is conjugated to a chelating moiety, selected from the group consisting of: TMT (6,6″-bis[N,N″,N′″-tetra(carboxymethyl)aminomethyl)-4′-(3-amino-4-methoxyphenyl)-2,2′:6′,2″-terpyridine), DOTA (1, 4,7,10-tetraazacyclododecane-NN′,N″(N′″-tetraacetic acid), TCMC, DO3A, CB-DO2A, NOTA, Diamsar, DTPA, CHX-A″-DTPA, TETE, Te2A, HBED, DFO, DFOsq, DFO-NCS and HOPO or other chelating agent known to the skilled person.

28. The olaratumab of claim 27, wherein the radiolabelled olaratumab is 89Zr-olaratumab or a ⁸⁹Zr labelled olaratumab, wherein the ⁸⁹Zr is conjugated to the olaratumab via a linker, such as ⁸⁹Zr-DFO-olaratumab, ⁸⁹Zr-DFO-NCS-olaratumab, or ⁸⁹Zr-DFO-Sq-olaratumab.

29. A method for in vivo imaging or detection of a PDGFRalpha-expressing cancer in a subject, wherein the method comprises:
administering to a subject in need thereof, a radiolabelled-olaratumab antibody or antigen binding fragment thereof, preferably an olaratumab antibody or antigen binding fragment thereof of any one of claims 3 to 28,
detecting the antibody or antigen binding fragment thereof in the subject,
thereby imaging or detecting the cancer in the subject.

30. A method for the diagnosis of a PDGFRalpha-expressing cancer in a subject, where the method comprises:
administering to a subject in need thereof, a radiolabelled-olaratumab antibody or antigen binding fragment thereof, preferably an olaratumab antibody or antigen binding fragment thereof of any one of claims 3 to 28,
determining the presence or absence of the antibody or antigen binding fragment thereof in the subject,
thereby diagnosing the cancer in the subject.

31. The method of claim 29 or 30, wherein the detection of the antibody or fragment, or the presence or absence of the antibody or fragment is compared to a background or standard level to determine the presence of the cancer, thereby imaging or detecting or diagnosing the cancer in the subject.

32. The method of claim 29 or 30, wherein the detection or the presence or absence is compared to a model of disease classification or disease progression that is derived from data from one or more individuals, thereby imaging or detecting or diagnosing the cancer in the subject.

33. A method for producing an image of a PDGFRalpha-expressing cancer in a subject, the method comprising:
administering to a subject suspected of having the cancer, a radiolabelled-olaratumab antibody or antigen binding fragment thereof, preferably an olaratumab antibody or antigen binding fragment thereof of any one of claims 3 to 28,
detecting the antibody or antigen binding fragment thereof in the subject,
thereby producing an image of the cancer.

34. A method for producing an image of a PDGFRalpha-expressing cancer, the method comprising:
infusing an effective amount of a radiolabelled-olaratumab antibody or antigen binding fragment thereof, preferably an olaratumab antibody or antigen binding fragment thereof of any one of claims 3 to 28,
detecting the antibody or antigen binding fragment thereof,
thereby producing an image of the cancer.

35. The method of claim 33 or 34, wherein the image of the cancer produced is used to generate training data for development of a model of disease progression or disease classification, optionally wherein the model is a machine learning model based on neural networks.

36. A method for classifying a cancer as sensitive to treatment with an inhibitor of PDGFRalpha, the method comprises:
administering to a subject in need thereof, a radiolabelled-olaratumab antibody or antigen binding fragment thereof, preferably an olaratumab antibody or antigen binding fragment thereof of any one of claims 3 to 28,
detecting the antibody or antigen binding fragment thereof in the subject,
whereby the cancer is classified as sensitive to treatment with an inhibitor of PDGFRalpha when detection of the antibody or fragment thereof is below or the same as a background or threshold level; or
whereby the cancer is classified as not being sensitive to treatment with an inhibitor of PDGFRalpha when detection of the antibody or fragment thereof is above a background or threshold level.

37. A method for classifying a cancer as sensitive to treatment with an inhibitor of PDGFRalpha, the method comprises:
administering to a subject in need thereof, a radiolabelled-olaratumab antibody or antigen binding fragment thereof, preferably an olaratumab antibody or antigen binding fragment thereof of any one of claims 3 to 28,
detecting the antibody or antigen binding fragment thereof in the subject,
whereby the cancer is classified as sensitive to treatment with an inhibitor of PDGFRalpha based on the output of a deep learning algorithm, indicating the presence of PDGFRalpha expressing cancer cells in the subject,
whereby the cancer is not classified as sensitive to treatment with an inhibitor of PDGFRalpha based on the output of a deep learning algorithm, indicating the absence of PDGFRalpha expressing cancer cells in the subject.

38. A method for classifying or selecting a patient for eligibility for cancer therapy with an inhibitor of PDGFRalpha, the method comprising:
administering to a subject in need thereof, a radiolabelled-olaratumab antibody or antigen binding fragment thereof, preferably an olaratumab antibody or antigen binding fragment thereof of any one of claims 3 to 28,
detecting the antibody or antigen binding fragment thereof in the subject,
whereby the patient is classified/selected for cancer therapy with an inhibitor of PDGFRalpha when detection of the antibody or fragment thereof is above a background or standard level;
whereby the patient is not classified/selected for cancer therapy with an inhibitor of PDGFRalpha when detection of the antibody or fragment thereof is the same or below a background or standard level.

39. A method for classifying or selecting a patient for eligibility for cancer therapy with an inhibitor of PDGFRalpha, the method comprising:
administering to a subject in need thereof, a radiolabelled-olaratumab antibody or antigen binding fragment thereof, preferably an olaratumab antibody or antigen binding fragment thereof of any one of claims 3 to 28,
detecting the antibody or antigen binding fragment thereof in the subject,
whereby the patient is classified/selected for cancer therapy with an inhibitor of PDGFRalpha based on the output of a deep learning algorithm, indicating the presence of PDGFRalpha expressing cancer cells in the subject,
whereby the patient is not classified/selected for cancer therapy with an inhibitor of PDGFRalpha based on the output of a deep learning algorithm, indicating the absence of PDGFRalpha expressing cancer cells in the subject.

40. A method for determining the response to treatment of a PDGFRalpha-expressing cancer or a subject having a PDGFRalpha-expressing cancer, the method comprising:
administering to a subject who has received or is receiving a treatment for a PDGFRalpha-expressing cancer, a radiolabelled-olaratumab antibody or antigen binding fragment thereof, preferably an olaratumab antibody or antigen binding fragment thereof of any one of claims 3 to 28,
detecting the antibody or antigen binding fragment thereof in the subject,
comparing the amount of antibody or antigen binding fragment thereof detected in the subject compared to a reference amount of the antibody or antigen binding fragment thereof detected in the subject prior to the treatment,
determining that the cancer has responded to the treatment when the amount of the antibody or antigen binding fragment thereof detected is lower than the reference amount,
determining that the cancer has not responded to the treatment when the amount of the antibody or antigen binding fragment thereof is the same or higher than the reference amount,
thereby determining the response to treatment of a PDGFRalpha-expressing cancer or a subject having a PDGFRalpha-expressing cancer.

41. The method of claim 40, wherein the treatment received or being received by the subject is selected from: surgery, chemotherapy, immunotherapy (such as CAR-T therapy), radiotherapy (including external beam radiation or immuno-radiotherapy) an inhibitor of PDGFRalpha and/or combinations thereof.

42. The method of any one of claims 33 to 39 or 41, wherein the inhibitor of PDGFRalpha is an olaratumab antibody, or functional antigen binding fragment thereof.

43. The method of claim 42, wherein the olaratumab antibody comprises a heavy variable chain as defined in SEQ ID NO: 4 and a light variable chain as defined in SEQ ID NO: 12.

44. Use of a radiolabelled-olaratumab antibody or antigen binding fragment thereof, preferably an olaratumab antibody or antigen binding fragment thereof of any one of claims 3 to 28, in the manufacture of a medicament for:
in vivo imaging or detection of a PDGFRalpha-expressing cancer;
diagnosis of a PDGFRalpha-expressing cancer;
producing an image of a PDGFRalpha-expressing cancer;
classifying a cancer as sensitive to treatment with an inhibitor of PDGFRalpha; or
classifying or selecting a patient for eligibility for cancer therapy with an inhibitor of PDGFRalpha.

45. The method of any one of claims 29 to 43, wherein the method comprises allowing the radiolabelled-olaratumab antibody or antigen binding fragment thereof to concentrate at sites and/or tissues in said subject where the PDGFRalpha antigen is found in the subject, prior to the step of detecting.

46. The method of any one of claims 29 to 43, wherein detection of the radiolabelled-olaratumab antibody or antigen binding fragment thereof comprises Positron Emission Tomography (PET), SPECT imaging or combinations thereof.

47. The method of any one of claims 29 to 43, or the use of claim 44, wherein a subject in need thereof is a subject that is suspected of having or is considered at risk of having a PDGFRalpha-expressing cancer.

48. The method of any one of claims 29 to 43, or the use of claim 44, wherein the cancer that is detected, imaged, diagnosed, classified or selected is selected from the group consisting of: soft tissue sarcoma (STS), chondrosarcoma, leiomyosarcoma, liposarcoma, osteosarcoma, and rhabdomyosarcoma.

49. The method of any one of claims 29 to 43, or the use of claim 44, wherein the cancer that is detected, imaged, diagnosed, classified or selected is selected from the group consisting of: gastrointestinal stromal tumour (GIST), non-small cell lung cancer (NSCLC), pancreatic cancer, hepatocellular carcinoma, breast cancer, neuroendocrine tumours, ovarian cancer, cervical cancer, uterine cancer (such as adenosquamous carcinoma of the uterine cervix), colorectal cancer, gastric cancer, salivary gland cancer, biliary cancer, glioma, prostate cancer and renal cell carcinoma.


SEQUENCE LISTING (20 sequences)

SEQ ID NO: 1 - skipped

SEQ ID NO: 2 - protein, 16 aa, synthetic construct
SFFYTGSTYYNPSLRS

SEQ ID NO: 3 - protein, 17 aa, synthetic construct
QSTYYYGSGNYYGWFDR

SEQ ID NO: 4 - protein, 127 aa, synthetic construct
QLQLQESGPGLVKPSETLSLTCTVSGGSINSSSYYWGWLRQSPGKGLEWIGSFFYTGSTYYNPSLRSRLTISVDTSKNQFSLMLSSVTAADTAVYYCARQSTYYYGSGNYYGWFDRWDQGTLVTVSS

SEQ ID NO: 5 - protein, 30 aa, synthetic construct
QLQLQESGPGLVKPSETLSLTCTVSGGSIN

SEQ ID NO: 6 - protein, 16 aa, synthetic construct
WGWLRQSPGKGLEWIG

SEQ ID NO: 7 - protein, 32 aa, synthetic construct
RLTISVDTSKNQFSLMLSSVTAADTAVYYCAR

SEQ ID NO: 8 - protein, 11 aa, synthetic construct
WDQGTLVTVSS

SEQ ID NO: 9 - protein, 11 aa, synthetic construct
RASQSVSSYLA

SEQ ID NO: 10 - protein, 7 aa, synthetic construct
DASNRAT

SEQ ID NO: 11 - protein, 9 aa, synthetic construct
QQRSNWPPA

SEQ ID NO: 12 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPAFGQGTKVEIK

SEQ ID NO: 13 - protein, 23 aa, synthetic construct
EIVLTQSPATLSLSPGERATLSC

SEQ ID NO: 14 - protein, 15 aa, synthetic construct
WYQQKPGQAPRLLIY

SEQ ID NO: 15 - protein, 32 aa, synthetic construct
GIPARFSGSGSGTDFTLTISSLEPEDFAVYYC

SEQ ID NO: 16 - protein, 10 aa, synthetic construct
FGQGTKVEIK

SEQ ID NO: 17 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 107 aa, synthetic construct [sequence omitted]

SEQ ID NO: 19 - protein, 457 aa, synthetic construct
QLQLQESGPGLVKPSETLSLTCTVSGGSINSSSYYWGWLRQSPGKGLEWIGSFFYTGSTYYNPSLRSRLTISVDTSKNQFSLMLSSVTAADTAVYYCARQSTYYYGSGNYYGWFDRWDQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPAFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC